(19) United States
(12) Patent Application Publication
(10) Pub. No.: US 2016/0002733 A1
(43) Pub. Date: Jan. 7, 2016

(54) ASSESSING RISK FOR ENCEPHALOPATHY INDUCED BY 5-FLUOROURACIL OR CAPECITABINE

(71) Applicant: THE BOARD OF TRUSTEES OF THE LELAND STANFORD JUNIOR UNIVERSITY, Palo Alto, CA (US)

(72) Inventor: Gilbert Chu, Stanford, CA (US)

(21) Appl. No.: 14/769,961

(22) PCT Filed: Feb. 26, 2014
(86) PCT No.: PCT/US14/18739

§ 371 Date: Aug. 24, 2015

Related U.S. Application Data
(60) Provisional application No. 61/772,949, filed on Mar. 5, 2013.

(51) Int. Cl. C12Q 1/68

(52) U.S. Cl. CPC C12Q 1/6886; C12Q 2600/156; C12Q 2600/106; C12Q 2600/142

(57) ABSTRACT

Methods and systems are provided for determining susceptibility to 5-fluorouracil (5-FU) or capecitabine toxicity. Methods are provided for treating a human subject based on a determined susceptibility to 5-fluorouracil (5-FU) or capecitabine toxicity.

### STATEMENT REGARDING FEDERALLY SPONSORED RESEARCH

This invention was made with Government support under Grant Number TR000093 awarded by the National Institute of Health. The Government has certain rights in the invention.

### BACKGROUND

Mild cognitive impairment, a common complaint of cancer patients treated with chemotherapy, is often referred to as “chemobrain.” Mechanisms for cognitive impairment remain unknown, although investigators have proposed several hypotheses, including low efficiency efflux pumps, deficits in DNA repair, reduced antioxidant capacity, deregulation of the immune response, and reduced capacity for neural repair. Neuropsychological deficits have occurred in women with breast cancer after chemotherapy, and are more common after high doses than after standard doses. These deficits correlate with chemotherapy administration, and not with anxiety, depression or fatigue. Abnormal brain white matter organization, measured by magnetic resonance diffusion tensor imaging, occur in women after chemotherapy in association with cognitive impairment.

Severe cognitive impairment with hyperammonemia is a rare and potentially fatal complication of chemotherapy. The syndrome occurs in the absence of liver disease following treatment of hematological malignancies, or following treatment of solid organ malignancies with the pyrimidine analog 5-fluorouracil (5-FU). 5-fluorouracil (5-FU) and capecitabine (the oral prodrug of 5-FU) are among the most commonly used anticancer drugs, with roles in the treatment of head and neck, esophageal, gastric, pancreatic, colon, rectal, and breast cancers. In one report, after high dose continuous infusion 5-FU, sixteen of 280 patients (5.7%) suffered encephalopathy with hyperammonemia. Encephalopathy has also occurred after the oral 5-FU pro-drug capecitabine, but the case reports do not document plasma ammonia levels.

Encephalopathy with hyperammonemia associated with 5-FU infusion has been reported as a rare complication, but a large fraction of patients may suffer from mild to moderate encephalopathy. Such patients may experience less severe nonspecific symptoms of fatigue, lethargy, and cognitive dysfunction interpreted as “chemobrain”. Moreover, the symptoms may resolve shortly after the last 5-FU or capecitabine dose, so that the patient appears to be healthy upon presenting for the next cycle of chemotherapy. Thus, mild to moderate encephalopathy after capecitabine is likely more common than currently appreciated.

Increased plasma ammonia levels have been used to make a diagnosis after a patient has already presented with frank encephalopathy. Methods to predict susceptibility to 5-FU and/or capecitabine toxicity can prevent morbidity as well as brain damage from repeated episodes of hyperammonemia and encephalopathy. The present invention provides methods and systems for determining susceptibility to 5-FU or capecitabine toxicity. The present invention also provides methods for treating a human subject based on a predicted susceptibility to 5-fluorouracil (5-FU) or capecitabine toxicity.

### PUBLICATIONS

Diasio R B, Beavers T L, Carpenter J T. Familial deficiency of dihydropyrimidine dehydrogenase. Biochemical basis for familial pyrimidinemia and severe 5-fluorouracil-induced toxicity. J Clin Invest. 81(1):47-51, 1988
Fantini M, Gianni L, Tassinari D, Nicoletti S, Possenti C, Drudi F et al. Toxic encephalopathy in elderly patients during treatment with capecitabine: literature review and a case report. J Oncol Pharm Pract. 17(3):288-291, 2011
Koenig H, Patel A. Biochemical basis for fluorouracil neurotoxicity. The role of Krebs cycle inhibition by fluoroacetate. Arch Neurol. 23(2):155-160, 1970
Milano G, Etienne M C, Pierrefite V, Barberi-Heyob M, Deporte-Fety R, Renee N. Dihydropyrimidine dehydrogenase deficiency and fluorouracil-related toxicity. Br J Cancer. 79(3-4):627-630, 1999
Niemann B, Rochlitz C, Herrmann R, Pless M. Toxic encephalopathy induced by capecitabine. Oncology. 66(4):331-335, 2004
Owen O E, Kalhan S C, Hanson R W. The key role of anaplerosis and cataplerosis for citric acid cycle function. J Biol Chem. 277(34):30409-30412, 2002
Patel A, Koenig H. Some neurochemical aspects of fluorocitrate intoxication. J Neurochem. 18(4):621-628, 1971
Strauss K A, Puffenberger E G, Morton D H. Maple Syrup Urine Disease. 1993
Videnovic A, Semenov I, Chua-Adajar R, Baddi L, Blumenthal D T, Beck A C et al. Capecitabine-induced multifocal leukoencephalopathy: a report of five cases. Neurology. 65(11):1792-1794; discussion 1685, 2005
Yeh K H, Cheng A L. High-dose 5-fluorouracil infusional therapy is associated with hyperammonaemia, lactic acidosis and encephalopathy. Br J Cancer. 75(3):464-465, 1997

### SUMMARY

Methods and systems are provided for determining a susceptibility to 5-fluorouracil (5-FU) or capecitabine toxicity in a human subject. Embodiments of the methods include assaying a biological sample from a human subject who has been diagnosed with cancer for the presence of a deleterious polymorphism or mutation in one or more of the genes listed in Tables 1 and 2. In some embodiments, a biological sample is assayed for the presence of a deleterious polymorphism or mutation in two or more of the genes listed in Tables 1 and 2 (e.g., ETFA and SLC25A2). In some embodiments, a biological sample is assayed for the presence of a deleterious polymorphism or mutation in all of the genes listed in Table 1. In some embodiments, assaying includes sequencing a nucleic acid isolated or amplified from a biological sample.

The methods further include: determining that a subject has an increased susceptibility to 5-fluorouracil (5-FU) or capecitabine toxicity when a deleterious polymorphism or mutation is present in a biological sample from the subject, or determining that a subject has a lack of increased susceptibility to 5-fluorouracil (5-FU) or capecitabine toxicity when a deleterious polymorphism or mutation is absent in a biological sample from the subject. In some embodiments, the methods include providing an analysis indicating whether an increased susceptibility was determined.

In some embodiments, the methods include directing a therapeutic intervention based on an analysis of susceptibility by the methods of the invention, comprising administration of an altered dose (e.g., a reduced dose) of 5-FU or capecitabine relative to the dose that would have been administered in the absence of such an analysis (i.e., an otherwise conventional dose). In some embodiments, the methods include directing a therapeutic intervention that does not comprise administration of 5-FU or capecitabine. In other words, in some embodiments, the methods include directing a therapeutic intervention that comprises a therapy other than administration of 5-FU or capecitabine. In some embodiments, the methods include directing a therapeutic intervention comprising administering 5-FU or capecitabine to the subject, measuring the level of ammonia in the blood, and monitoring for clinical signs of 5-FU toxicity (e.g., fatigue, lethargy, cognitive dysfunction, hyperammonemia and/or encephalopathy). Methods are also provided for treating a human subject based on a predicted susceptibility to 5-fluorouracil (5-FU) or capecitabine toxicity.

As demonstrated herein, capecitabine/fluorouracil urea-cycle encephalopathy is more common than currently believed. Thus, physicians (e.g., oncologists) that administer 5-FU or capecitabine should monitor plasma ammonia levels.

Systems and kits are provided for determining a susceptibility to 5-FU or capecitabine toxicity in a human subject. Suitable systems include: (i) a genotype determination element for determining the presence or absence in a biological sample of a deleterious polymorphism or mutation in one or more of the genes listed in Tables 1 and 2; and (ii) a prognosis analysis element for guiding a course of treatment based on the determined presence or absence of a deleterious polymorphism or mutation.

### BRIEF DESCRIPTION OF THE DRAWINGS

FIGS. 1A-1B Pathways associated with hyperammonemia. Mitochondrial steps occur inside the dotted lines. Key enzymes are shown in boxes. Mutated genes in Patient 1 are marked with stars. (FIG. 1A) Pathways for ammonia clearance. The urea cycle and pyrimidine biosynthesis remove ammonia. Key enzymes are: CPS I and CPS II, carbamoyl phosphate synthases type I and type II; NAGS, N-acetylglutamate synthase; ORNT, ornithine transporters SLC25A15 (ORNT1), SLC25A2 (ORNT2) and SLC25A29 (ORNT3); RR, ribonucleotide reductase; TS, thymidylate synthase. Other abbreviations: DHO, dihydroorotate; NAG, N-acetylglutamate; OMP, orotidine monophosphate; VPA, valproic acid. (FIG. 1B) Pathways for Krebs cycle anaplerosis. Anaplerosis replenishes intermediates in the Krebs cycle. Key enzymes (with subunits and/or components in parentheses): ACAD, acyl-CoA dehydrogenase (ACADVL, ACADVM, HADHA, HADHB, ETFA, ETFB, ETFDH); ACAS, acyl-CoA synthase family member ACSM2A; AST, aspartate transaminase; GLUD1, glutamate dehydrogenase 1; carnitine shuttle (CPT1, CPT2, SLC25A20, SLC22A5, MLYCD); MUT, methylmalonyl-CoA mutase (MMAA, MMAB, MMACHC, MMADHC); PC, pyruvate carboxylase; PCC, propionyl-CoA carboxylase (PCCA, PCCB, HLCS); PD, pyruvate dehydrogenase (PDHA1, DLAT). See Table 1 for subunit and component abbreviations.

FIGS. 2A-2C Patients with abnormal ammonia metabolism. (FIG. 2A) Slow ammonia clearance in Patient 1. The graph shows plasma ammonia levels as a function of days from her first dose of capecitabine, which was administered for 14 days (black bar). Lactulose was administered for 3 days (gray bar). The dotted line indicates the upper range of normal. (FIG. 2B) Elevated urine orotic acid after allopurinol challenge in Patient 1. The graph shows the urine orotic acid levels after challenge with 300 mg allopurinol. The peak urine orotic acid level was 16.5 nmol/mol creatinine. Normal for adult women (4.6±2.8 nmol/mol creatinine) is indicated by the dashed line, with the standard deviation marked in gray. (FIG. 2C) Plasma ammonia levels after capecitabine in prospectively enrolled patients. Plasma ammonia levels were measured at baseline (light gray bars) and at mid-cycle (dark gray bars). The peak level for Patient 24 may have been higher, because he forgot to donate blood until 2 days after completing the 14 day course of capecitabine. Patients appear in order of their mean baseline plasma ammonia levels. Statistically significant increases in mid-cycle compared to baseline levels occurred for 5 patients with p<0.01 (*) or p<0.001 (**).

FIG. 3 RNA-Seq analysis of SLC7A7 splice donor site mutation. Patient 1 was homozygous for a mutation at splice donor site SD-2 in SLC7A7, corresponding to the change, (A/C)AG|GUPuAGU>(A/C)GG|GUPuAGU. To determine whether this mutation affected the RNA, we analyzed RNA sequencing data from 12 acute myelogenous leukemia samples (numbers 1-12) that were heterozygous for five SNPs in SLC7A7 RNA, including the SD-2 splice donor site SNP found in Patient 1 at position 1083 (green). The x-axis shows the RNA position of the five SNPs. The y-axis shows the fraction of RNA-Seq reads for the five SNPs. The data show that the SD-2 splice donor SNP has no effect on the SLC7A7 RNA.

FIG. 4 Standard deviation vs. mean baseline ammonia. Each point represents one of the patients in the study. The line represents the linear fit to the data. Based on the slope of the linear fit, we estimated the standard deviation to be 25% of the mean baseline ammonia level for each patient.

FIG. 5 Normal DPYD enzymatic activity in Patient 1. Dihydropyrimidine dehydrogenase (DPYD) activity was measured in peripheral blood lymphocytes from Patient 1 and an age-matched healthy control. Samples were harvested at the same time, shipped on dry ice and analyzed by the laboratory of Dr. Robert Diasio (Mayo Clinic, Rochester, Minn.).

FIG. 6 lists measured plasma levels of amino acids in patient 1.

FIG. 7 lists measured levels of urine organic acids in patient 1.

FIG. 8 Missense or splicing site mutations in Patient 1. Allele frequencies and disease associations were obtained from the SNP database, SNP GeneView, GeneCards and the Protein database. Abbreviations: NA, not available; NV, normal variant based on SIFT and PolyPhen2 predictions and high allele frequency; Ref DNA, reference DNA sequence; SA, splice acceptor; SD, splice donor. Notes: A, T1406N was reported to be associated with low plasma arginine levels, but this association was not confirmed in a follow-up study. The patient's plasma arginine levels were abnormally elevated, ruling out any clinical effect due to T1406N; B, G159C shows decreased activity in cells transfected with a cDNA expression vector; C, P520L is predicted to preserve protein function and is not among the 64 mutations found in neonatal or severe infantile carnitine palmitoyltransferase II deficiency. P520L is not listed in the SNP database, and presumably rare; D, A499T confers normal enzymatic activity; E, T171I affects thermal stability of the ETF enzyme and is over-represented among patients with very-long-chain acyl-CoA dehydrogenase deficiency; F, The splice donor-2 polymorphism had no effect on RNA, as determined by RNA-seq analysis of AML data (FIG. 3); G, Patient 1 had normal enzymatic activity (FIG. 5).

FIGS. 9A-9C Genes with nonsense mutations. Average number of reads 84, range 6-498. Asterisks (*) indicate the maximum number of homozygous reads in SNPs adjacent to the nonsense mutation.

FIG. 10 Mutations at invariant splice sites in Patient 1. The table shows genes with mutations in the splice donor (SD) invariant GT, or splice acceptor site (SA) invariant AG. Asterisks (*) indicate the maximum number of homozygous reads in SNPs adjacent to the nonsense mutation.

FIG. 11 Indels in Patient 1. The Table shows indels sequenced more than once. Indels for CLCA4, SMARCA2, and ATN1 occur in repeated amino acid sequences, and are therefore presumed to be polymorphisms. ALMS1 is mutated in Alstrom syndrome and required for normal function of primary cilia. Knockdown of ALMS1 led to stunted cilia, and cells lacked the ability to increase calcium influx in response to mechanical stimuli.

FIG. 12 Deleterious mutations in Patient 1. The table shows the four deleterious mutations relevant for hyperammonemia. Patient 1 was heterozygous for each mutation.

FIG. 13 Deleterious SNPs among 44 hyperammonemia genes. Among the 44 hyperammonemia genes, 21 (in the table) contained SNPs deemed “deleterious” by SIFT and “damaging” by Polyphen. SNPs rs10891314 in DLAT and rs7104156 in PC are known to be non-pathogenic (NP) (GeneCards). Allele frequencies were not available (NA) and thus rare for 13 SNPs. The maximum allele frequency (max allele freq) was known for 16 genes, and unknown (x) for 5 genes.

FIG. 14 Frequency of deleterious SNPs in the population. The maximum allele frequency of the deleterious SNPs in FIG. 12 is known for 16 of the genes, and unknown for 5 genes. For the latter 5 genes, we assigned several values (Column 1) to the maximum allele frequency (Max allele freq, x): 0.0; 0.005, half the median; 0.010, the median; and 0.020, twice the median of the known values for the 16 genes. The sum of the maximum allele frequencies for the 21 genes (Column 2) represents the average number of deleterious SNPs in the population, which was then used as the Poisson parameter λ. The Poisson distribution estimates the probability for n deleterious SNPs, P(n)=(λⁿ/n!) exp(−λ). Columns 3-6 show the estimated fraction of the population carrying: zero, P(0); one or more, P(≧1); two or more, P(≧2); and three or more, P(≧3), deleterious SNPs.

### DETAILED DESCRIPTION

Methods and systems are provided for determining a susceptibility to 5-fluorouracil (5-FU) or capecitabine toxicity in a human subject.

5-FU and “Capecitabine” are chemotherapeutic agents commonly used in the treatment of head and neck, esophageal, gastric, pancreatic, colon, rectal, and breast cancers. As used herein, the term “5-FU” refers to any form of 5-FU, encompassing any and all compounds (e.g., drugs) that are converted into 5-FU in the body (i.e., 5-FU pro-drugs, e.g., capecitabine). For example, capecitabine, pentyl[1-(3,4-dihydroxy-5-methyltetrahydrofuran-2-yl)-5-fluoro-2-oxo-1H-pyrimidin-4-yl]carbamate, is an orally-administered pro-drug that is enzymatically converted to 5-FU in the body. The term “5-FU” encompasses the term “capecitabine.”

The term “susceptibility” is used herein to refer to the likelihood of being affected, or a tendency to be affected, by a condition of interest. For example, a subject who has an increased susceptibility to cancer has a higher likelihood of being diagnosed with cancer than someone who does not have an increased susceptibility to cancer. As is illustrated above, the term “susceptibility” is a relative term (e.g., relative to a control subject, an average subject of the population, a subject without cancer, a subject who does not harbor a deleterious polymorphism or mutation in any of the genes listed in Tables 1 and 2, etc.). As used herein, when a first subject has an “increased susceptibility to 5-fluorouracil (5-FU) or capecitabine toxicity,” the subject has an increased sensitivity to 5-FU such that at the same dose of 5-FU administered to a second subject who does not have an increased susceptibility, the administered 5-FU is more likely to be toxic to the first subject. In other words, a subject who has an “increased susceptibility to 5-fluorouracil (5-FU) or capecitabine toxicity” is more “sensitive” to 5-FU toxicity than someone who does not have an increased susceptibility and is thus more likely to suffer from 5-FU toxicity (e.g., at an equivalent dose). Likewise, when a first subject lacks an “increased susceptibility to 5-fluorouracil (5-FU) or capecitabine toxicity,” the subject does not have an increased sensitivity to 5-FU. In other words, a subject with a “lack of increased susceptibility to 5-fluorouracil (5-FU) or capecitabine toxicity” is not more “sensitive” to 5-FU and is thus not more likely to suffer from 5-FU toxicity.

As used herein, the term “otherwise conventional dose” is used in the context of a determination that a subject has an increased susceptibility to 5-FU or capecitabine toxicity. In some such cases, a therapeutic intervention is directed that comprises administration of a reduced dose of 5-FU or capecitabine. The reduced dose is reduced relative to the dose that would have been administered if the subject did not have an increased susceptibility to 5-fluorouracil (5-FU) or capecitabine toxicity (i.e., an otherwise conventional dose). Methods of determining an “otherwise conventional dose” (i.e., appropriate dose when a subject has not been determined to have an increased susceptibility to 5-FU toxicity) of 5-FU or capecitabine are known in the art and depend on various factors including (but not limited to) age, weight, stage and type of cancer, etc.

The term “toxicity” as used herein refers to any negative effects (e.g., symptoms), which may or may not be life-threating. For example, 5-FU toxicity encompasses chemotherapy-associated cognitive impairment, which is sometimes referred to as “chemobrain.” As “chemobrain” can be an indication of encephalopathy, 5-FU toxicity encompasses nonspecific symptoms (e.g., fatigue, lethargy, and cognitive dysfunction) in addition to more specific symptoms (e.g., hyperammonemia and/or encephalopathy). All of the above symptoms can be used as a readout of 5-FU toxicity. For example, a patient who experiences hyperammonemia, encephalopathy, fatigue, lethargy, cognitive dysfunction, and/or a combination thereof after being administered with 5-FU can be considered have suffered from 5-FU toxicity. A subject with an increased susceptibility to 5-FU or capecitabine toxicity is more likely to experience a symptom of 5-FU toxicity (e.g., hyperammonemia, encephalopathy, fatigue, lethargy, cognitive dysfunction, and/or a combination thereof) than a subject without an increased susceptibility.

Clinical signs of 5-FU or capecitabine toxicity can include (but are not necessarily limited to): hyperammonemia, encephalopathy, fatigue, lethargy, cognitive dysfunction, and/or a combination thereof. In some embodiments, 5-FU or capecitabine toxicity is detected by an increase in plasma ammonia levels (i.e., hyperammonemia). As is known in the art, a plasma ammonia level ranging up to about 50 μmol/L (micromole per liter) is considered “normal.” Accordingly, “hyperammonemia” as used herein refers to a plasma level of ammonia that is above about 30 μmol/L. Methods of measuring the level of ammonia in the blood (e.g., plasma) are known in the art and any convenient technique can be used. For non-limiting examples of suitable techniques see Howanitz et al., Clin Chem 1984; 30:906-8: Influences of specimen processing and storage conditions on results for plasma ammonia; and Maranda et al., Clin Biochem 2007; 40:531-5: false positives in plasma ammonia measurement and their clinical impact in a pediatric population; both of which are hereby incorporated by reference in their entirety. In some embodiments, 5-FU or capecitabine toxicity is detected by the presence of encephalopathy. In some embodiments, the clinical signs of 5-FU or capecitabine toxicity include fatigue, lethargy, cognitive dysfunction, and/or a combination thereof. As is known in the art, clinical signs of cognitive dysfunction include: confusion, disorientation, reduced balance, reduced coordination, slurred speech, reduced responsiveness, ataxia, and/or a combination thereof.

The term “assaying” is used herein to include the physical steps of manipulating a biological sample to generate data related to the sample. As will be readily understood by one of ordinary skill in the art, a biological sample must be “obtained” prior to assaying the sample. Thus, the term “assaying” implies that the sample has been obtained. The terms “obtained” or “obtaining” as used herein encompass the physical extraction or isolation of a biological sample from a subject. The terms “obtained” or “obtaining” as used herein also encompasses the act of receiving an extracted or isolated biological sample. For example, a testing facility can “obtain” a biological sample in the mail (or via delivery, etc.) prior to assaying the sample. In some such cases, the biological sample was “extracted” or “isolated” (and thus “obtained”) from the subject by a second entity prior to mailing, and then “obtained” by the testing facility upon arrival of the sample. Thus, the testing facility can obtain the sample and then assay the sample, thereby producing data related to the sample. Alternatively, a biological sample can be extracted or isolated from a subject by the same person or same entity that subsequently assays the sample.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. “Assaying for the presence of” can be determining the amount of something present and/or determining whether it is present or absent.

As referred to in the subject methods, “assaying” a sample (e.g., a biological sample from a subject) for the presence of a deleterious polymorphism or mutation means performing an assay to determine whether a polymorphism or mutation is present. Subsequently, if a polymorphism or mutation is present, the polymorphism or mutation is assessed for whether it is deleterious (see details below). The term “assay” refers to any method of determination. Examples of assays to determine whether a deleterious polymorphism or mutation is present include, but are not limited to: hybridization methods (e.g., array hybridization of nucleic acid from the biological sample, or amplified from the biological sample, to an array of nucleic acids (e.g., SNP microarrays); in situ hybridization; in situ hybridization followed by FACS; Dynamic allele-specific hybridization (DASH) genotyping; SNP detection through molecular beacons; and the like); single strand conformation polymorphism assay; Temperature gradient gel electrophoresis assay; Denaturing high performance liquid chromatography (DHPLC); High Resolution Melting analysis; enzyme-based methods (e.g., restriction fragment length polymorphism (RFLP) detection); PCR-based methods (e.g., Flap endonuclease (FEN) based assays, 5′-nuclease assay (e.g. TaqMan assay), and the like); nucleic acid sequencing methods (e.g., Sanger sequencing, Next Generation sequencing (i.e., massive parallel high throughput sequencing, e.g., Illumina's reversible terminator method, Roche's pyrosequencing method (454), Life Technologies' sequencing by ligation (the SOLiD platform), Life Technologies' Ion Torrent platform, single molecule sequencing, etc.)); etc.

Examples of some of the sequencing methods above are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39) and Morozova (Genomics. 2008 92:255-64), which are incorporated by reference for the general descriptions of the methods and the particular steps of the methods, including all starting products, reagents, and final products for each of the steps.

In some embodiments, both alleles for a particular base position are determined and it is therefore determined whether the subject is homozygous or heterozygous at the particular base. In some embodiments, the determination is made as to whether a polymorphism or mutation (e.g., a deleterious polymorphism or mutation) is present, but it is not determined whether the subject is homozygous or heterozygous at the particular base.

In some embodiments, the biological sample can be assayed directly. In some embodiments, nucleic acid of the biological sample is amplified (e.g., by PCR) prior to assaying. As such, techniques such as PCR (Polymerase Chain Reaction), RT-PCR (reverse transcriptase PCR), qRT-PCR (quantitative RT-PCR, real time RT-PCR), etc. can be used prior to the hybridization methods and/or the sequencing methods discussed above.

A polymorphism or mutation can be detected in DNA and/or RNA. As is known in the art, an mRNA sequence can be a direct reflection of DNA sequence because mRNA is transcribed from the DNA. Thus, DNA and/or mRNA is a suitable nucleic acid for “assaying” in any of the subject methods. For example, detecting an “A” at base 112 of an mRNA transcript reveals that an “A” is present at that corresponding position in the DNA (“A” on the non-template strand, i.e., coding strand; and “T” on the template strand, i.e., non-coding strand).

The term “nucleic acid” includes DNA, RNA (double-stranded or single stranded), analogs (e.g., PNA or LNA molecules) and derivatives thereof. The terms “ribonucleic acid” and “RNA” as used herein mean a polymer composed of ribonucleotides. The terms “deoxyribonucleic acid” and “DNA” as used herein mean a polymer composed of deoxyribonucleotides. The term “mRNA” means messenger RNA. An “oligonucleotide” generally refers to a nucleotide multimer of about 10 to 100 nucleotides in length, while a “polynucleotide” includes a nucleotide multimer having any number of nucleotides.

The term “polymorphism” (e.g., a single nucleotide polymorphism (SNP)) as used herein refers to an allele (e.g., a nucleotide, or base pair) at a specific location in the genome that is present in the organism's population (e.g., a human population) at a particular frequency. The allele frequency for a polymorphism of interest may be known or unknown and the polymorphism may be new or it may be a previously identified polymorphism. The term “mutation” as used herein refers to any base pair that is different than a known reference sequence. Thus, the term mutation encompasses the term polymorphism, but it is possible for a mutation to not be a polymorphism. For example, a mutation made in the laboratory that does not exist in a subject in a population is a mutation that is not a polymorphism. A mutation that is identified from a human patient can be considered a polymorphism since the mutation therefore exists in the population (even if it only exists in the one patient). A polymorphism of interest can be a known mutation that exists in the population at a particular frequency. A polymorphism of interest can be a mutation that is known to associate with a particular phenotype (e.g., a disease state; a non-disease state; a trait, e.g., eye color; susceptibility to a disease; susceptibility to an adverse reaction, e.g. an adverse reaction to a particular medication or treatment, etc.). In some cases, the polymorphism of interest is known, but has not previously been associated with a disease. A polymorphism can be a mutation that has not been previously described or a mutation that has been previously described. A polymorphism or mutation of interest can be any mutation (e.g., an insertion, a deletion, a base pair substitution, a translocation, an inversion, etc.). The term “polymorphism or mutation” is used herein to encompass both terms.

As used herein, the term “deleterious polymorphism or mutation” means deleterious to the activity of the encoded protein (i.e., a polymorphism or mutation that indicates altered activity of the encoded protein, damaged activity of the encoded protein, etc.). Accordingly a deleterious polymorphism or mutation may be found in the sequence encoding the protein, and/or in sequences that affect the expression, stability, or translation of the RNA transcript (e.g., promoter, enhancer, or silencing sequences; sequences that control or affect intron splicing, e.g., splice donor and/or splice acceptor sequences; sequences in the 5′ or 3′ untranslated region (i.e., 5′ UTR, 3′ UTR) that affect stability or translation; etc.). In some embodiments, a deleterious polymorphism or mutation is found in the nucleic acid sequence encoding the protein. In some embodiments, a deleterious polymorphism or mutation changes the amino acid sequence of the encoded protein (relative to the fully functional protein) such that the encoded protein has reduced activity (e.g., a loss of function mutation, a mutation that reduces the stability of the protein, etc.). In some cases, the encoded protein has 95% or less (e.g., 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 0%) of the activity of the fully functional protein. In some embodiments, a deleterious polymorphism or mutation changes the amino acid sequence of the encoded protein (relative to the fully functional protein) such that the encoded protein has an increased activity (e.g., a gain of function mutation, a mutation that increases the stability of the protein, etc.). In some cases, the encoded protein has 10% or more (e.g., 15% or more, 20% or more, 50% or more, 60% or more, 75% or more, 85% or more, 90% or more, 100% or more, 150% or more, 200% or more, 250% or more, or 300% or more) increased activity relative to the normal, non-altered (i.e., reference) protein.

In some embodiments, a deleterious polymorphism or mutation alters at least one of the encoded amino acids. However, not all polymorphisms or mutations that alter an amino acid of the encoded protein are deleterious. For example, a non-deleterious polymorphism or mutation may alter the amino acid sequence such that the encoded protein exhibits increased activity (e.g., due to greater enzymatic activity, enhanced stability, etc.). As such, a polymorphism or mutation that that alters one or more amino acids of the encoded protein is deleterious if the newly encoded protein has decreased overall activity.

There are numerous ways to assess whether a polymorphism or mutation is deleterious. In some cases, a polymorphism or mutation is assessed by performing a functional assay (e.g. a binding assay, an enzymatic assay, etc., depending on the function of the protein) comparing the activity of a protein encoded by the original sequence to the activity of the protein encoded by the altered sequence. Such assays can be performed in vitro (e.g., using purified components or cellular extracts; in living cells in culture; etc.) or in vivo. In some cases, a polymorphism or mutation is assessed in silico. For example, suitable programs include, but are not limited to (a) the SIFT (Sorting Tolerant From Intolerant) algorithm, which assumes that important positions in the amino acid sequence of a protein have been conserved during evolution and predicts the effects of substitutions at each position in the amino acid sequence; (b) the PolyPhen-2 (Polymorphism Phenotyping version 2) algorithm, which uses sequence-based and structure-based algorithms to predict the functional importance of an amino acid substitution. One of ordinary skill in the art will be familiar with suitable programs. Publications describing the in silico assessment of polymorphisms or mutations include: Kumar et al., Predicting the effects of coding non-synonymous variants on protein function using the SIFT algorithm. Nat Protoc 2009; 4:1073-81; Adzhubei et al., A method and server for predicting damaging missense mutations. Nat Methods 2010; 7:248-9; all of which are hereby specifically incorporated by reference. In some cases, the polymorphism or mutation has previously been assessed (for whether it is a deleterious polymorphism or mutation) and this information can be found in patent and/or non-patent (i.e., scientific) literature.

A “biological sample” as used herein can be any sample from (e.g., extracted from, collected from, isolated from, etc.) a subject (e.g., a mammalian subject, a human subject, etc.). The term “biological sample” encompasses a clinical sample, and also includes any tissue (e.g., tissue obtained by surgical resection, tissue obtained by biopsy, etc.), any cell, any cells in culture, cell supernatants, cell lysates, tissue samples, organs, bone marrow, whole blood, fractionated blood, plasma, serum, hair, skin, and the like. In some cases, cells, fluids, or tissues derived from a subject are cultured, stored, or manipulated prior to assaying. In some instances, a biological sample is a tissue sample (e.g., a biopsy, whole blood, fractionated blood, plasma, serum, saliva, hair, skin, cheek swab, and the like) or is extracted from a tissue sample (e.g., a composition comprising nucleic acid). Examples of biological samples include, but are not limited to cell and tissue cultures derived from a subject (and derivatives thereof, such as supernatants, lysates, and the like); tissue samples and body fluids; non-cellular samples (e.g., column eluants; acellular biomolecules such as proteins, lipids, carbohydrates, nucleic acids; synthesis reaction mixtures; nucleic acid amplification reaction mixtures; in vitro biochemical or enzymatic reactions or assay solutions; or products of other in vitro and in vivo reactions, etc.); etc. A biological sample can be extracted, isolated, or collected from a subject by any convenient means (e.g., blood draw, biopsy collection, cheek swab, etc.)

The present invention provides methods of treating a human subject based on predicted susceptibility of the subject to 5-fluorouracil (5-FU) or capecitabine toxicity.

The terms “treatment”, “treating”, “treat” and the like are used herein to generally refer to obtaining a desired pharmacologic and/or physiologic effect. “Treatment” as used herein covers any treatment of a disease in a mammal, particularly a human, and includes: (a) inhibiting the disease symptom, i.e., arresting development of the disease and/or symptom(s) related to the disease; or (b) relieving the disease symptom, i.e., causing regression of the disease or symptom(s) related to the disease. This is need of treatment include those diagnosed with cancer. In some embodiments, the cancer is head and neck, esophageal, gastric, pancreatic, colon, rectal, and/or breast cancer.

An “effective amount” is an amount sufficient to effect beneficial or desired clinical results. An effective amount can be administered in one or more administrations. For purposes of this invention, an effective amount of a compound (e.g., 5-FU, a 5-FU prodrug, a compound other than 5-FU, etc.) is an amount that is sufficient to palliate, ameliorate, stabilize, reverse, prevent, slow or delay the progression of (and/or symptoms associated with) the disease state (e.g., cancer).

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of biological sample was used and/or how it was used, the technique used to assay the sample, the results of the assay (e.g., the number and/or identity of any determined polymorphisms or mutations), the assessment as to whether any determined polymorphisms or mutations are deleterious polymorphisms or mutations (as defined above), information as to how the polymorphisms or mutations were assessed to determine whether they are deleterious, a statement describing if an increased susceptibility (or a lack of increased susceptibility) to 5-fluorouracil (5-FU) or capecitabine toxicity was determined, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

### Methods

The subject methods concern determination of susceptibility to 5-fluorouracil (5-FU) or capecitabine toxicity. The administration of 5-FU (e.g., a 5-FU prodrug) is a commonly used therapeutic intervention for cancer. Thus, the subject methods can be used to determine whether a patient with cancer can and/or should be treated with 5-FU. As such, the subject methods can be used to evaluate the level of risk of toxicity associated with 5-FU treatment. However, because 5-FU toxicity is independent of the presence or absence of a cancer diagnosis, any subject is a suitable subject for the provided methods. Thus, the subject methods can be used for determining the susceptibility (e.g., increased susceptibility; lack of increased susceptibility) of any subject (without regard to a cancer diagnosis) to 5-fluorouracil (5-FU) or capecitabine toxicity. In some embodiments, the subject is a subject who has been diagnosed with cancer. In other words, in some cases, the subject methods are useful for determining the susceptibility (e.g., increased susceptibility; lack of increased susceptibility) of a cancer patient (i.e., a subject diagnosed with cancer) to 5-fluorouracil (5-FU) or capecitabine toxicity.

In some embodiments, the methods include providing an analysis indicating whether an increased susceptibility was determined. As described above, an analysis can be an oral or written report (e.g., written or electronic document). The analysis can be provided to the subject, to the subject's physician, to a testing facility, etc. The analysis can also be accessible as a website address via the internet. In some such cases, the analysis can be accessible by multiple different entities (e.g., the subject, the subject's physician, a testing facility, etc.).

5-FU is toxic and is detoxified in the liver by a process involving dihydropyrimidine dehydrogenase (“DPYD” or “DPD”). As is known in the art, the detoxification of 5-FU is compromised in a patient with DPYD deficiency (e.g., caused by the presence of a deleterious polymorphism or mutation in DPYD). Thus, a patient with a DPYD deficiency who receives a standard or conventional dose of 5-FU effectively responds as if they received a higher dose. Thus, in some cases the dosage of 5-FU administered can be reduced when the patient has a DPYD deficiency. Accordingly, in some embodiments, in addition to being assayed for the presence of a deleterious polymorphism or mutation in one or more of the genes listed in Tables 1 and 2, a biological sample is assayed for DPYD enzymatic activity (e.g., to determine whether the level of activity falls within what is considered by those of ordinary skill in the art to be the normal range) and/or assayed for the presence of a deleterious polymorphism or mutation in DPYD. Any convenient assay for DPYD enzymatic activity may be used and examples of suitable assays are known in the art.

In some embodiments, in addition to determining a susceptibility to 5-FU toxicity, the methods further include directing a therapeutic intervention. In some cases (e.g., when a lack of increased susceptibility to 5-fluorouracil (5-FU) or capecitabine toxicity is determined), a suitable therapeutic intervention includes the administration of 5-FU. In some cases (e.g., when an increased susceptibility to 5-fluorouracil (5-FU) or capecitabine toxicity is determined), a suitable therapeutic intervention does not include the administration of 5-FU. In other words, in some cases, a suitable therapeutic intervention is any convenient therapeutic intervention (e.g., use of a drug other than 5-FU, irradiation therapy, etc.) other than the administration of 5-FU. A therapeutic intervention other than the administration of 5-FU (or a 5-FU prodrug) includes any convenient method of therapy appropriate to the situation (e.g., appropriate for the patient, appropriate for the diagnosis, etc.).

In some embodiments, the methods include, when an increased susceptibility to 5-fluorouracil (5-FU) or capecitabine toxicity is determined, directing a therapeutic intervention comprising administration of a reduced dose of 5-FU or capecitabine relative to an otherwise conventional dose (described above).

Administration of 5-FU (and/or prodrugs thereof), including the determination of dosing, is known in the art. For example, see Twelves et al., Ann Oncol. 2012 May; 23(5):1190-7. While the prodrug capecitabine is administered orally, 5-FU (i.e., 5-FU/folinic acid (FA)) is generally administered by bolus i.v. Although the determination of dosing and route of administration are known in the art for 5-FU and 5-FU prodrugs, known methods do not take into account susceptibility to toxicity as disclosed herein. Thus, in some embodiments, the administration of 5-FU is altered (e.g., decreased dose, reduced frequency, etc.) for a subject for whom an increased susceptibility to 5-fluorouracil (5-FU) or capecitabine toxicity has been determined.

In some embodiments, after determining an increased susceptibility to 5-fluorouracil (5-FU) or capecitabine toxicity, a therapeutic intervention that includes the administration of 5-FU is directed. In some such cases, the level of ammonia in the blood of the subject are measured at regular intervals before and after administration of the 5-FU and order to monitor blood (e.g., plasma) levels of ammonia. When levels of ammonia are too high (e.g., hyperammonemia), then 5-FU administration can be stopped or reduced (e.g., reduced dose, reduced frequency, etc.). When levels of ammonia are low, 5-FU administration may be increased (e.g., increased dose, increased frequency, etc.). Accordingly, by monitoring the subject's blood (e.g., plasma) ammonia levels, the dosage and/or frequency of 5-FU administration can be custom tailored (i.e., optimized) for the subject such that the benefits of 5-FU treatment may be realized without resulting in 5-FU toxicity.

In some embodiments, the methods include monitoring the subject for clinical signs of 5-FU or capecitabine toxicity (described above). The inventors demonstrate in the examples below that capecitabine/fluorouracil urea-cycle encephalopathy is more common than currently believed. Thus, physicians (e.g., oncologists) that administer 5-FU or capecitabine should monitor plasma ammonia levels. If clinical signs of 5-FU toxicity are observed, the subject can be treated appropriately, as would be known by one of ordinary skill in the art (e.g., lactulose treatment, rifaximin treatment, phenylbutyrate treatment, and the like) to bring down the levels of plasma ammonia. Lactulose increases fecal nitrogen excretion and acidifies the stool to prevent ammonia absorption. Rifaximin alters the gut flora. Phenylbutyrate increases urinary excretion of nitrogen. Treatment to bring down the levels of plasma ammonia (e.g., using Lactulose, Rifaximin, and/or Phenylbutyrate) can prevent progressive brain damage and permit continuation of the chemotherapy regimen.

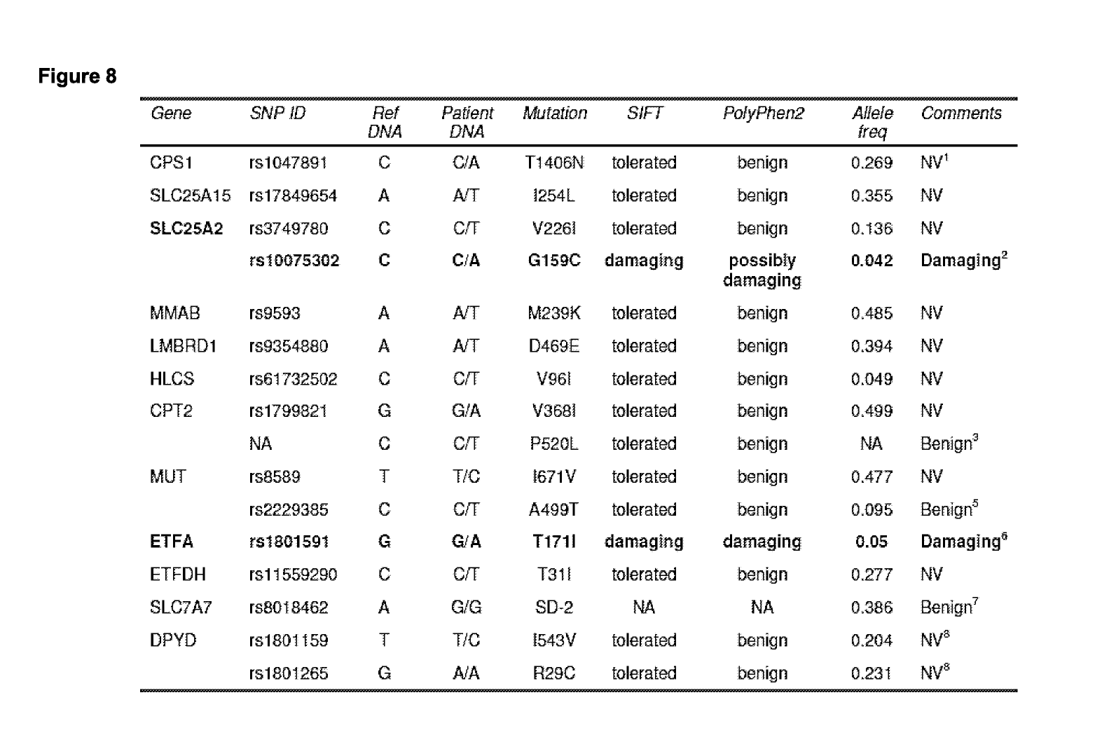

In some embodiments, a biological sample is assayed for the presence of a deleterious polymorphism or mutation in any of the genes listed in Tables 1 and 2. Examples of specific alleles and amino acid substitutions that can be assayed for can be found in FIGS. 8-13.

In some embodiments, a biological sample is assayed for the presence of a deleterious polymorphism or mutation in the gene ETFA (electron-transfer-flavoprotein alpha polypeptide), which links acyl-CoA dehydrogenase to the respiratory chain. In some embodiments the deleterious polymorphism or mutation is the A allele of the polymorphic marker rs1801591, which results in the T171I mutation (threonine to isoleucine at amino acid position 171) in ETFA (see FIG. 8). In some embodiments, a biological sample is assayed for the presence of a deleterious polymorphism or mutation in the gene SLC25A2 (solute carrier family 25 member 2), encoding the ornithine transporter ORNT2. In some embodiments the deleterious polymorphism or mutation is the A allele of the polymorphic marker rs10075302, which results in the G159C mutation (glycine to cysteine at amino acid position 159) in SLC25A2 (see FIG. 8). In some embodiments, a biological sample is assayed for the presence of a deleterious polymorphism or mutation in the gene ACSM2A (acetyl-CoA synthetase family member 2A), which activates medium chain fatty acids for beta-oxidation by forming a thioester with CoA (thus, ACSM2A participates in a pathway associated with hyperammonemia). In some embodiments the deleterious polymorphism or mutation is the nonsense mutation R115* in ACSM2A, which generates a 462 amino acid truncation in the 577 amino acid protein. In some embodiments, a biological sample is assayed for the presence of a deleterious polymorphism or mutation in the gene ALMS1 (Alstrom Syndrome protein). In some embodiments the deleterious polymorphism or mutation is the L525_T527 del/insP mutation (indel mutation) in ALMS1, which replaces L525, E526, and T527 with proline.

In some embodiments, a biological sample is assayed for the presence of a deleterious polymorphism or mutation in one or more (e.g., 2 or more, 3 or more, 4 or more, 5 or more, 6 or more, 7 or more, 8 or more, etc.) of the genes listed in Tables 1 and 2 (e.g., ETFA and/or SLC25A2). In some embodiments, a biological sample is assayed for the presence of a deleterious polymorphism or mutation in one or more (e.g., 2 or more, 3 or more, 4 or more, 5 or more, 6 or more, 7 or more, 8 or more, etc.) of the genes listed in Table 1 (e.g., ETFA and/or SLC25A2). In some embodiments, a biological sample is assayed for the presence of a deleterious polymorphism or mutation in two or more (e.g., 3 or more, 4 or more, 5 or more, 6 or more, 7 or more, 8 or more, etc.) of the genes listed in Tables 1 and 2 (e.g., ETFA and SLC25A2). In some embodiments, a biological sample is assayed for the presence of a deleterious polymorphism or mutation in two or more (e.g., 3 or more, 4 or more, 5 or more, 6 or more, 7 or more, 8 or more, etc.) of the genes listed in Table 1 (e.g., ETFA and SLC25A2). In some embodiments, a biological sample is assayed for the presence of a deleterious polymorphism or mutation in all of the genes listed in Table 1.

The genes listed in Table 1 are genes that are known to associate with hyperammonemia, and include genes involved in primary hyperammonemia as well as genes involved in secondary hyperammonemia (see working examples below). The genes listed in Table 2 are genes that also associate with hyperammonemia because they contribute to Krebs cycle anaplerosis (e.g., they are involved in the Krebs cycle, fatty acid oxidation, or organic acidemia), the process that replenishes the Krebs cycle intermediates, α-ketoglutarate, succinyl-CoA and oxaloacetate. As such, deleterious polymorphisms or mutations in any of the genes listed in Tables 1 and 2 result in increased ammonia levels, and therefore increase the susceptibility of a subject to 5FU or capecitabine toxicity. In some embodiments, a biological sample from a human subject is assayed for the presence of a deleterious polymorphism or mutation in one or more of the genes listed in Tables 1 and 2. In some embodiments, a biological sample from a human subject is assayed for the presence of a deleterious polymorphism or mutation in a hyperammonemia gene, a gene involved in the urea cycle, a gene involved in Krebs cycle anaplerosis, a gene involved in fatty acid oxidation, and/or a gene involved in organic acidemia (see Tables 1 and 2). In all of the embodiments in this paragraph, an increased susceptibility to 5-FU or capecitabine toxicity is determined when a deleterious polymorphism or mutation is present in the biological sample.

### Reagents, Systems, and Kits

Also provided are reagents, systems and kits thereof for practicing one or more of the above-described methods. The subject reagents, systems and kits thereof may vary greatly. Reagents of interest include reagents specifically designed for use in determining a susceptibility to 5-fluorouracil (5-FU) or capecitabine toxicity in a human subject. The term system refers to a collection of reagents, however compiled, e.g., by purchasing the collection of reagents from the same or different sources. The term kit refers to a collection of reagents provided, e.g., sold, together.

One type of such reagent is a genotype determination element. A genotype determination element provides for assaying a biological sample for the presence or absence of deleterious polymorphism or mutation (or multiple polymorphisms or mutations) of interest (e.g., in one or more of the genes listed in Tables 1 and 2). One non-limiting example of a suitable genotype determination element is a genotyping array of probe nucleic acids in which SNPs (single nucleotide polymorphisms) of the determinative genes of interest (e.g., one or more of the genes listed in Tables 1 and 2) are represented. A variety of different array formats are known in the art, with a wide variety of different probe structures, substrate compositions and attachment technologies. In some embodiments, the arrays include probes for one or more polymorphisms or mutations in one or more (e.g., two or more, three or more, four or more, five or more, ten or more, fifteen or more, twenty or more, thirty or more, forty or more, or all) of the genes listed in Tables 1 and 2.

Another non-limiting example of a suitable genotype determination element is an array of primer pairs for amplifying one or more (e.g., two or more, three or more, four or more, five or more, ten or more, fifteen or more, twenty or more, thirty or more, forty or more, or all) of the genes (or any fragment thereof) listed in Tables 1 and 2. In some cases, the primers are specifically designed to detect SNPs at known polymorphic positions. In some cases, the primers are specifically designed to amplify the entire gene of interest (or fragment thereof) such that the presence or absence of a known or unknown deleterious polymorphism or mutation can be determined from the amplicon (e.g., by sequencing the amplicon).

Where the subject arrays and/or primer pair sets include probes (or primer pairs) for additional genes (e.g., those not listed in Tables 1 and 2), in certain embodiments the number of additional genes that are represented and are not directly or indirectly related to determining a susceptibility to 5-FU toxicity does not exceed about 50%, and usually does not exceed about 25%. In certain embodiments where additional genes are included, a great majority of genes in the collection are listed in Tables 1 and 2, where by great majority is meant at least about 75%, usually at least about 80% and sometimes at least about 85, 90, 95% or higher, including embodiments where 100% of the genes in the collection are listed in Table 1 or Table 2.

The systems and kits of the subject invention may include an above-described genotype determination element (e.g., arrays, gene specific primer collections, etc.). The systems and kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

The subject systems and kits can also include a prognosis analysis element, which element is, in many embodiments, a reference or control genotype (e.g., database of known polymorphisms and/or mutations and their associated frequencies in various populations) that can be employed, e.g., by a suitable computing means, to make a prognostic determination (e.g. determine whether a subject has an increased susceptibility to 5-FU toxicity) based on the determined presence or absence of a deleterious polymorphism or mutation that has been determined with the above described genotype determination element. One non-limiting example of a prognosis analysis element includes a database of allele frequencies (frequencies of various deleterious or non-deleterious alleles/polymorphisms/mutations). Such frequencies can be used as a control or reference in determining whether a subject with a deleterious polymorphism or mutation has an increased susceptibility relative to a control population.

An exemplary suitable system includes (i) a genotype determination element for determining the presence or absence in a biological sample of a deleterious polymorphism or mutation in one or more of the genes listed in Tables 1 and 2; and (ii) a prognosis analysis element for guiding a course of treatment based on the determined presence or absence of a deleterious polymorphism or mutation.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, flash drive, CD, etc., on which the information has been recorded. Yet another means that may be present is a website address which may be used via the internet to access the information at a removed site. Any convenient means may be present in the kits.

The invention now being fully described, it will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Hyperammonemia Genes are Involved in the Urea Cycle or Pathways that Affect the Urea Cycle

Table 1 shows 45 genes associated with hyperammonemia. 41 genes were identified by searching OMIM (Online Mendelian Inheritance in Man) with the keyword “hyperammonemia”, and then reviewing the literature to confirm a genuine association with hyperammonemia. The list of genes was augmented by adding two mitochondrial membrane transporters for ornithine and citrulline (SLC25A2 (ORNT2) and SLC25A29 (ORNT3)), which encode ornithine transporters that act in parallel with the classical urea cycle ornithine transporter SLC25A15 (ORNT1). The Table was further augmented by adding two genes (ACSM2A and ACSM2B), which encode acetyl-CoA synthetase family members 2A and 2B. ACSM2A and ACSM2B were added to Table 1 because a deleterious polymorphism was identified in ACSM2A in a patient (see below). ACSM2A and ACSM2B participate in a pathway associated with hyperammonemia.

TABLE 1
Genes associated with hyperammonemia. Diseases and disease categories
are underlined. Square brackets enclose the protein function and the specific disease. For
some genes, the protein function and associated disease are derived directly from the gene
name.

| | | |
|---|---|---|
| Gene | # | Mutation |

Hyperammonemia - Urea Cycle Defect

| | | |
|---|---|---|
| ALDH18A1 | 1 | aldehyde dehydrogenase 18 family member A1 [ornithine, arginine, proline |
| | | biosynthesis, cutis laxa type IIIA] |
| ARG1 | 2 | arginase, liver [argininemia] |
| ASS1 | 3 | argininosuccinate synthase 1 [citrullinemia type I] |
| ASL | 4 | argininosuccinate lyase [argininosuccinic aciduria] |
| CPS1 | 5 | carbamoyl phosphate synthase 1, mitochondrial |
| GLUL | 6 | glutamate-ammonia ligase [synthesis of glutamine from glutamate, congenital |
| | | glutamine deficiency] |
| NAGS | 7 | N-acetylglutamate synthase |
| OTC | 8 | ornithine transcarbamylase |
| SLC7A7 | 9 | solute carrier family 7 (cationic amino acid transporter, y+ system) member 7 |
| | | [Arg, Lys, ornithine transport in kidney and small intestine, lysinuric protein |
| | | intolerance] |
| SLC25A2 | 10 | solute carrier family 25 (mitochondrial carrier; ornithine transporter) member 2 |
| | | [ORNT2] |
| SLC25A13 | 11 | solute carrier family 25 member 13 (citrin) [exchange of Asp for Glu across |
| | | inner mitochondrial membrane, citrullinemia type II] |
| SLC25A15 | 12 | solute carrier family 25 (mitochondrial carrier; ornithine transporter) member 15 |
| | | [ORNT1, hyperornithinemia-hyperammonemia-homocitrullinuria (HHH) |
| | | syndrome] |
| SLC25A29 | 13 | solute carrier family 25 (mitochondrial carnitine/acylcarnitine carrier protein |
| | | CACL) member 29 [ORNT3] |

Hyperammonemia - Krebs Cycle Defect

| | | |
|---|---|---|
| DLAT | 14 | dihydrolipoamide S-acetyltransferase [in mitochondrial complex that converts |
| | | pyruvate to acetyl-CoA] |
| GLUD1 | 15 | glutamate dehydrogenase 1 [mitochondrial deamination of glutamate to alpha- |
| | | ketoglutarate, hyperinsulinism-hyperammonemia syndrome] |
| PC | 16 | pyruvate carboxylase [mitochondrial pyruvate oxidation to oxaloacetate] |
| PDHA1 | 17 | pyruvate dehydrogenase (lipoamide) alpha 1 [in mitochondrial complex that |
| | | converts pyruvate to acetyl-CoA] |
| TUFM | 18 | Tu translation elongation factor, mitochondrial [protein translation in |
| | | mitochondria, combined oxidative phosphorylation deficiency] |

Hyperammonemia - Organic Acidemia

| | | |
|---|---|---|
| HLCS | 19 | holocarboxylase synthase (biotin-(propionyl-CoA-carboxylase (ATP- |
| | | hydrolysing)) ligase) [gluconeogenesis, branched chain amino acid catabolism] |
| HMGCL | 20 | 3-hydroxymethyl-3-methylglutaryl-CoA lyase [final step in leucine degradation] |
| IVD | 21 | isovaleryl-CoA dehydrogenase [valine, leucine, isoleucine degradation, |
| | | isovaleric acidemia] |
| LMBRD1 | 22 | LMBR1 domain containing 1 [cobalamin transporter, homocystinuria- |
| | | megaloblastic anemia type F] |
| MCCC1 | 23 | methylcrotonoyl-CoA carboxylase 1 (alpha) [leucine catabolism] |
| MCCC2 | 24 | methylcrotonoyl-CoA carboxylase 2 (beta) [leucine catabolism] |
| MLYCD | 25 | malonyl-CoA-decarboxylase [stimulates fatty acid oxidation by converting |
| | | malonyl-CoA to acetyl-CoA] |
| MMAA | 26 | methylmalonic aciduria (cobalamin deficiency) cblA type [methylmalonic |
| | | aciduria] |
| MMAB | 27 | methylmalonic aciduria (cobalamin deficiency) cblB type [methylmalonic |
| | | aciduria] |
| MMACHC | 28 | methylmalonic aciduria (cobalamin deficiency) cblC type, with homocystinuria |
| | | [methylmalonic aciduria] |
| MMADHC | 29 | methylmalonic aciduria (cobalamin deficiency) cblD type, with homocystinuria |
| | | [methylmalonic aciduria] |
| MUT | 30 | methylmalonyl CoA mutase [isomerization of methylmalonyl-CoA to succinyl- |
| | | CoA, methylmalonic aciduria] |
| PCCA | 31 | propionyl CoA carboxylase, alpha polypeptide [propionic acidemia] |
| PCCB | 32 | propionyl CoA carboxylase, beta polypeptide [propionic acidemia] |

Hyperammonemia - Mitochondrial Fatty Acid Oxidation Defect

| | | |
|---|---|---|
| ACADVL | 33 | acyl-CoA dehydrogenase, very long chain |
| ACADM | 34 | acyl-CoA dehydrogenase, C-4 to C-12 straight chain |
| CPT1A | 35 | carnitine palmitoyltransferase 1A (liver) |
| CPT2 | 36 | carnitine palmitoyltransferase 2 |
| ETFA | 37 | electron-transfer-flavoprotein, alpha polypeptide [glutaric acidemia type IIA] |
| ETFB | 38 | electron-transfer-flavoprotein, beta polypeptide [glutaric acidemia type IIB] |
| ETFDH | 39 | electron-transferring-flavoprotein dehydrogenase [glutaric acidemia type IIC] |
| HADHA | 40 | hydroxyacyl-CoA dehydrogenase/3-ketoacyl-CoA thiolase/enoyl-CoA |
| | | hydratase, alpha subunit |
| HADHB | 41 | hydroxyacyl-CoA dehydrogenase/3-ketoacyl-CoA thiolase/enoyl-CoA |
| | | hydratase, beta subunit |
| SLC25A20 | 42 | solute carrier family 25 (carnitine/acylcarnitine translocase) member 20 |
| | | [carnitine cycle] |
| SLC22A5 | 43 | solute carrier family 22 (organic cation/carnitine transporter) member 5 |
| | | [carnitine deficiency] |

Other relevant genes

| | | |
|---|---|---|
| ACSM2A | 44 | acetyl-CoA synthetase A-anaplerosis pathway (Fatty Acid Oxidation) |
| ACSM2B | 45 | acetyl-CoA synthetase B-anaplerosis pathway (Fatty Acid Oxidation) |

TABLE 2
Additional gene products not previously associated with hyperammonemia
that can increase susceptibility to 5-FU and capecitabine toxicity when
defective (i.e., when the gene has a deleterious polymorphism or mutation).
Other relevant genes - Krebs Cycle anaplerosis pathways
(Krebs Cycle, Fatty Acid Oxidation, Organic Acidemia)

| Gene | # | Mutation |
|---|---|---|
| ACAA1 | 1 | acetyl-CoA acyltransferase 1 (Fatty acid oxidation) |
| ACAA2 | 2 | acetyl-CoA acyltransferase 2 (Fatty acid oxidation) |
| ACAS | 3 | acetyl-CoA synthetase A (Fatty acid oxidation) |
| ACADS | 4 | acyl-CoA dehydrogenase, C-2 to C-3 short chain (Fatty acid oxidation) |
| ACAD9 | 5 | acyl-CoA dehydrogenase family, member 9 (Fatty acid oxidation) |
| ACADL | 6 | acyl-CoA dehydrogenase, long chain (Fatty acid oxidation) |
| ACADSB | 7 | acyl-CoA dehydrogenase, short/branched chain (Fatty acid oxidation) |
| ACAD8 | 8 | acyl-CoA dehydrogenase family, member 8 (Fatty acid oxidation) |
| ACAD10 | 9 | acyl-CoA dehydrogenase family, member 10 (Fatty acid oxidation) |
| ACAD11 | 10 | acyl-CoA dehydrogenase family, member 11 (Fatty acid oxidation) |
| ACAT1 | 11 | acetyl-CoA acetyltransferase 1 (Fatty acid oxidation) |
| ACAT2 | 12 | acetyl-CoA acetyltransferase 2 (Fatty acid oxidation) |
| ACO1 | 13 | aconitase 1, soluble (Krebs cycle) |
| ACO2 | 14 | aconitase 2, mitochondrial (Krebs cycle) |
| AGPAT1 | 15 | 1-acylglycerol-3-phosphate O-acyltransferase 1 (Fatty acid oxidation) |
| AUH | 16 | AU RNA binding protein/enoyl-CoA hydratase (Fatty acid oxidation) |
| CPT1B | 17 | carnitine palmitoyltransferase 1B (Fatty acid oxidation) |
| CPT1C | 18 | carnitine palmitoyltransferase 1C (Fatty acid oxidation) |
| CS | 19 | citrate synthase (Krebs cycle) |
| DECR1 | 20 | 2,4-dienoyl CoA reductase 1, mitochondrial (Fatty acid oxidation |
| DECR2 | 21 | 2,4-dienoyl CoA reductase 2, peroxisomal (Fatty acid oxidation |
| ECH1 | 22 | enoyl CoA hydratase 1, peroxisomal (Fatty acid oxidation) |
| ECI1 | 23 | enoyl-CoA delta isomerase 1 (Fatty acid oxidation) |
| ECI2 | 24 | enoyl-CoA delta isomerase 2 (Fatty acid oxidation) |
| EHHADH | 25 | enoyl-CoA, hydratase/3-hydroxyacyl CoA dehydrogenase (Fatty acid |
| | | oxidation) |
| ECHS1 | 26 | enoyl CoA hydratase, short chain, 1, mitochondrial (Fatty acid oxidation) |
| FH | 27 | fumarate hydratase (Krebs cycle) |
| GOT1 | 28 | aspartate transaminase, glutamic-oxaloacetic transaminase 1, soluble |
| | | (AST, aspartate aminotransferase 1) (Krebs cycle) |
| GOT2 | 29 | aspartate transaminase, glutamic-oxaloacetic transaminase 2, |
| | | mitochondrial (AST, aspartate aminotransferase 2) (Krebs cycle) |
| HADH | 30 | hydroxyacyl-CoA dehydrogenase (Fatty acid oxidation) |
| IDH1 | 31 | isocitrate dehydrogenase 1 (NADP+), soluble (Krebs cycle) |
| IDH1 | 32 | isocitrate dehydrogenase 2 (NADP+), mitochondrial (Krebs cycle) |
| IDH3A | 33 | isocitrate dehydrogenase 3 (NAD+) alpha (Krebs cycle) |
| IDH3B | 34 | isocitrate dehydrogenase 3 (NAD+) beta (Krebs cycle) |
| IDH3G | 35 | isocitrate dehydrogenase 3 (NAD+) gamma (Krebs cycle) |
| MCEE | 36 | methylmalonyl CoA epimerase (Fatty acid oxidation, Organic acidemia) |
| MDH1 | 37 | malate dehydrogenase 1, NAD (soluble) (Krebs cycle) |
| MDH1B | 38 | malate dehydrogenase 1B, NAD (soluble) (Krebs cycle) |
| MDH2 | 39 | malate dehydrogenase 2, NAD (mitochondrial) (Krebs cycle) |
| ME1 | 40 | malic enzyme 1, NADP(+)-dependent, cytosolic (Krebs cycle) |
| ME2 | 41 | malic enzyme 2, NAD(+)-dependent, mitochondrial (Krebs cycle) |
| ME3 | 42 | malic enzyme 3, NADP(+)-dependent, mitochondrial (Krebs cycle) |
| OGDH | 43 | oxoglutarate (alpha-ketoglutarate) dehydrogenase (lipoamide) (Krebs |
| | | cycle) |
| OGDHL | 44 | oxoglutarate dehydrogenase-like (Krebs cycle) |
| PDHA2 | 45 | pyruvate dehydrogenase (lipoamide) alpha 2 (Krebs cycle) |
| PDHB | 46 | Pyruvate Dehydrogenase (lipoamide) beta (Krebs cycle) |
| SDHAF1 | 47 | succinate dehydrogenase complex assembly factor 1 (Krebs cycle) |
| SDHAF2 | 48 | succinate dehydrogenase complex assembly factor 2 (Krebs cycle) |
| SDHA | 49 | succinate dehydrogenase complex, subunit A, flavoprotein (Fp) (Krebs |
| | | cycle) |
| SDHB | 50 | succinate dehydrogenase complex, subunit B, iron sulfur (Ip) (Krebs |
| | | cycle) |
| SDHC | 51 | succinate dehydrogenase complex, subunit C, integral membrane |
| | | protein, 15 kDa (Krebs cycle) |
| SDHD | 52 | succinate dehydrogenase complex, subunit D, integral membrane |
| | | protein (Krebs cycle) |
| SUCLG1 | 53 | succinate-CoA ligase, alpha subunit (Krebs cycle) |
| SUCLA2 | 54 | succinate-CoA ligase, ADP-forming, beta subunit (Krebs cycle) |
| SUCLG2 | 55 | succinate-CoA ligase, GDP-forming, beta subunit (Krebs cycle) |

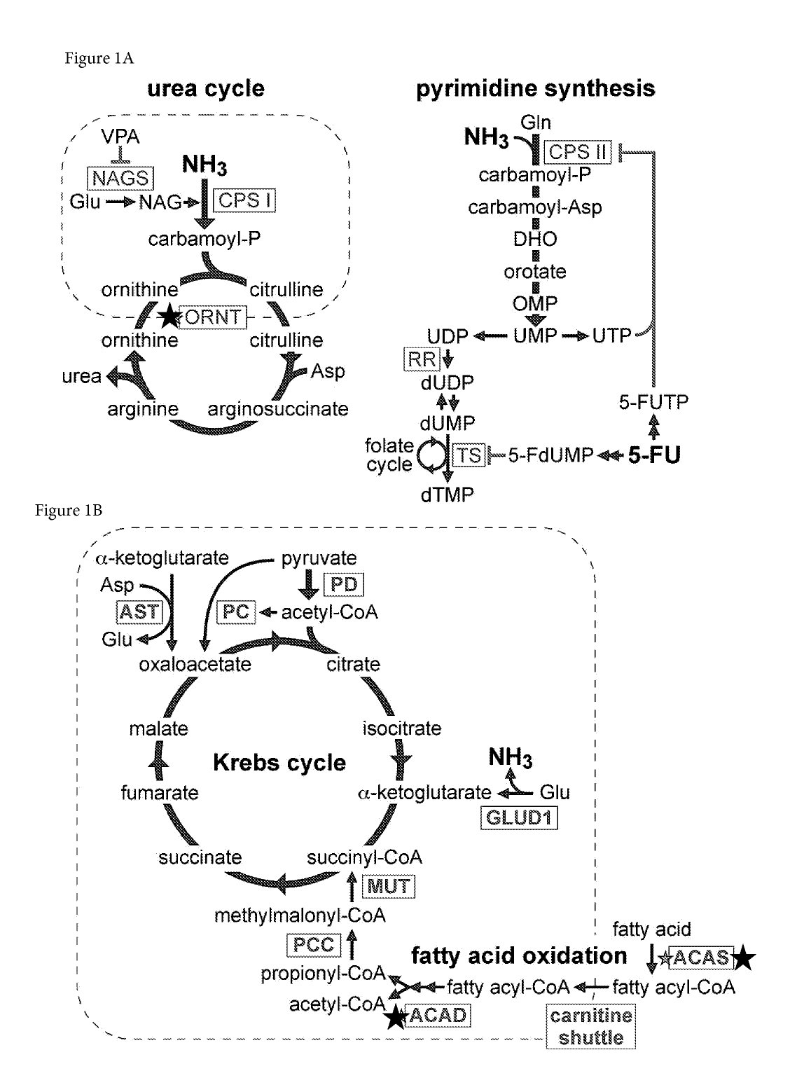

Primary hyperammonemia arises from mutations in the urea cycle (FIG. 1A). Secondary hyperammonemia arises from mutations in the Krebs cycle, mitochondrial fatty acid oxidation and organic acidemia genes (Table 1). We will discuss below how these secondary hyperammonemia genes facilitate anaplerosis, the process that replenishes the Krebs cycle intermediates, α-ketoglutarate, succinyl-CoA and oxaloacetate (FIG. 1B).

GLUD1 mutations, which cause hyperinsulinism-hyperammonemia syndrome, generate hyperactive GLUD1 by desensitizing glutamate dehydrogenase to allosteric inhibition by GTP. GLUD1 is the only hyperammonemia gene with autosomal dominant inheritance. Hyperactive GLUD1 increases ammonia by deamination of glutamate and secondary depletion of N-acetylglutamate, thus inhibiting the urea cycle (FIG. 1B). In response to glutamate depletion, aspartate transaminase (AST, GOT1, GOT2) activity increases (FIG. 1B), but AST competes with argininosuccinate synthase (ASS) for aspartate, inhibiting the urea cycle at a second point (FIG. 1A).

PD (PDHA1, PDHA2, PDHB) mutations decrease acetyl-CoA levels, down-regulating PC activity (FIG. 1B). Both PD and PC mutations disrupt conversion of pyruvate to oxaloacetate. Anaplerosis increases the conversion of α-ketoglutarate to oxaloacetate via AST (GOT1, GOT2), thus inhibiting the urea cycle by competing with ASS for aspartate.

Fatty acid oxidation, proprionic acidemia and methylmalonic acidemia mutations block the supply of succinyl-CoA to the Krebs cycle (FIG. 1B). Anaplerosis by a compensatory increase in GLUD1 activity explains the decreased glutamate and glutamine levels in patients with these acidemias, and inhibits the urea cycle as described for GLUD1 mutations. Propionic and methylmalonic acidemias also cause hyperammonemia independently of succinyl-CoA depletion. Propionic or methylmalonic acid injected into rats cause hyperammonemia with N-acetylglutamate depletion. Indeed, propionyl-CoA, which accumulates in propionic and methylmalonic acidemias, is a competitive inhibitor of N-acetylglutamate synthase, thus inhibiting the urea cycle. Furthermore, methylmalonyl-CoA, which accumulates in methylmalonic acidemia, is a competitive inhibitor of PC, inhibiting the urea cycle as described for PC mutations.

Mutations in a subset of the branched-chain amino acid degradation genes (HLCS, HMGCL, IVD, MCCC1, and MCCC2, but not the maple syrup urine disease genes, BCKDHA, BCKDHB, DBT, and DLD) cause hyperammonemia, probably due to accumulation of acyl-CoA intermediates of branched-chain amino acid degradation that inhibit pyruvate dehydrogenase (PD), inhibiting the urea cycle as described for PD mutations.

TUFM (Tu translation elongation factor, mitochondrial) mutations cause combined oxidative phosphorylation deficiency by reduced translation of mitochondrial proteins. Since oxidative phosphorylation is coupled to fatty acid oxidation and the Krebs cycle, mutations inhibit the urea cycle as described for mutations in those pathways. In conclusion, mutations cause hyperammonemia by disrupting the urea cycle either directly or indirectly via Krebs cycle anaplerosis.

Genes with roles in the urea cycle cause primary hyperammonemia, and genes with roles in the Krebs cycle, mitochondrial fatty acid oxidation, and organic acidemias cause secondary hyperammonemia. Despite their apparent diversity, the secondary hyperammonemia genes proved to facilitate anaplerosis, the process that replenishes the Krebs cycle intermediates, α-ketoglutarate, succinyl-CoA and oxaloacetate.

Krebs cycle anaplerosis inhibits the urea cycle by competition for glutamate and aspartate (FIG. 1). Glutamate undergoes conversion to α-ketoglutarate in the Krebs cycle, and to N-acetylglutamate in the urea cycle. Aspartate is a substrate for conversion of α-ketoglutarate to oxaloacetate in the Krebs cycle, and citrulline to arginosuccinate in the urea cycle.

To understand the effects of anaplerosis, consider the autosomal dominant GLUD1 mutations, which constitutively activate glutamate dehydrogenase to increase ammonia production via glutamate deamination, and inhibit ammonia elimination by decreasing the availability of glutamate for the urea cycle. Consider mutations in fatty acid oxidation and in the proprionic and methylmalonic acidemias, which block the supply of succinyl-CoA to the Krebs cycle. Anaplerosis by a compensatory increase in GLUD1 activity explains glutamate depletion in these patients. Consider mutations in PC (pyruvate carboxylase) and PD (pyruvate dehydrogenase), which block the supply of oxaloacetate to the Krebs cycle. Anaplerosis by a compensatory increase in AST activity decreases the availability of aspartate for the urea cycle.

In summary, hyperammonemia arises by direct or indirect suppression of the urea cycle.

### Methods

### Exome Sequencing

We sequenced the whole exome of Patient 1 to an average of 50-fold coverage (Hudson-Alpha Institute, Huntsville, Ala.). To determine if a particular amino acid substitution affects protein function, we utilized the SIFT and PolyPhen-2 algorithms. The SIFT (Sorting Tolerant From Intolerant) algorithm assumes that important positions in the amino acid sequence of a protein have been conserved during evolution, and predicts the effects of substitutions at each position in the amino acid sequence (29). PolyPhen-2 (Polymorphism Phenotyping version 2) algorithm uses sequence-based and structure-based algorithms to predict the functional importance of an amino acid substitution (30). Allele frequencies and other information for specific genes were obtained from GeneCards

### RNA Sequencing

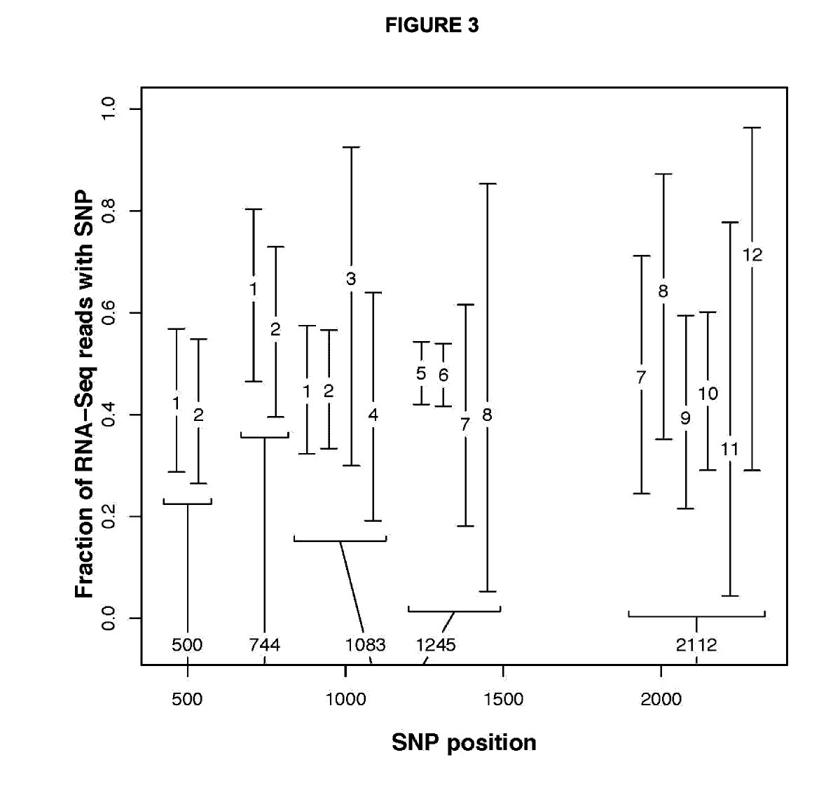

To determine whether a homozygous mutation in a splice donor site affected the RNA, we analyzed published RNA sequencing data from 12 acute myelogenous leukemia samples that were heterozygous for splice site mutation. The leukemia samples corresponded to samples labeled 1-12 in FIG. 3: SRR061899, SRR061823, SRR061886, SRR061900, SRR061757, SRR054844, SRR061824, SRR061898, SRR061897, SRR061758, SRR061885, SRR054845, respectively.

Prospective Measurement of Plasma Ammonia Levels in Patients Treated with Capecitabine

Patients donated whole blood for analysis after providing consent according to a protocol approved by the Stanford University Administrative Panel for the Protection of Human Subjects.

Plasma ammonia levels were obtained at Stanford University Medical Center, which followed a strict protocol of immediately placing the blood sample on ice, and then analyzing the sample within 15 minutes. Samples not placed on ice, or analyzed after a longer delay yield artificially elevated plasma ammonia levels due to release of ammonia from erythrocytes and deamination of plasma amino acids (52, 53).

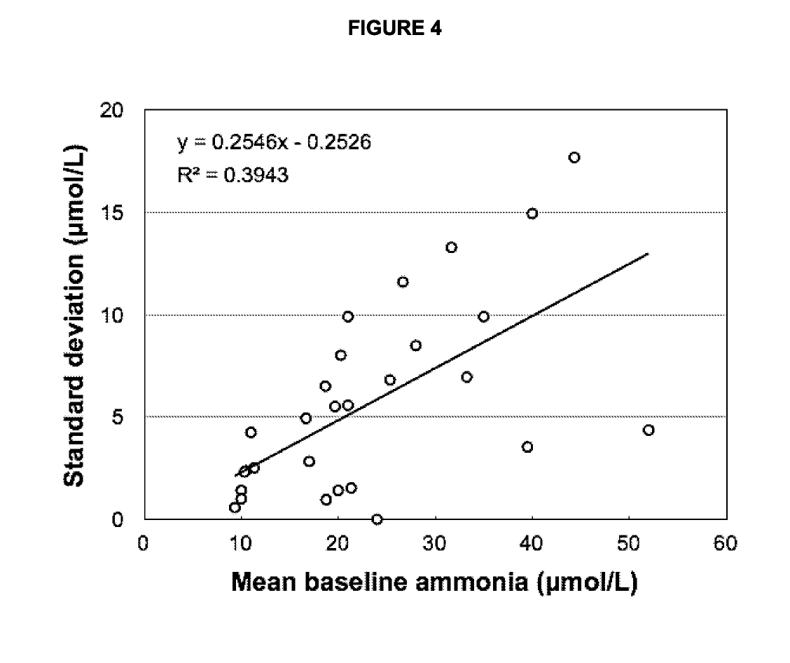

Baseline plasma ammonia levels were estimated from 2 and 4 measurements prior to initiating capecitabine, or at least 7 days after the last capecitabine dose. Errors for baseline levels were estimated to be 25% of the corresponding mean levels, based on a linear fit to the standard deviations plotted as a function of the mean levels for each patient (FIG. 4).

Mid-cycle levels were measured after patients had taken capecitabine for 7 to 14 days. Although mid-cycle levels required blood draws on days that patients did not have a clinic appointment, we obtained 2 mid-cycle samples from Patients 7, 16, and 24, and 3 mid-cycle samples from Patient 17. The average standard deviation of the mid-cycle levels for these four patients was 25%, matching the estimated error for the baseline values of all patients.

### Results

### Patient 1

A 67 y female with gastric adenocarcinoma underwent subtotal gastrectomy and Roux-en-Y gastrojejunostomy, followed by two cycles of adjuvant carboplatin and capecitabine (1000 mg/m² twice a day for 14 days), and then 50 Gy of radiation therapy to the tumor bed with concurrent capecitabine (1000 mg/m² twice a day). During each course of capecitabine, she experienced extreme lethargy, without mucositis, diarrhea or hand-foot syndrome.

On the third cycle of carboplatin and capecitabine, she self-administered folate 1 mg/d hoping to prevent lethargy. From days 5 to 14 of capecitabine, she became increasingly confused, and then combative and ataxic. Two days after the last capecitabine dose, she was taken to local emergency room for delirium and found to have a normal CT scan of the brain.

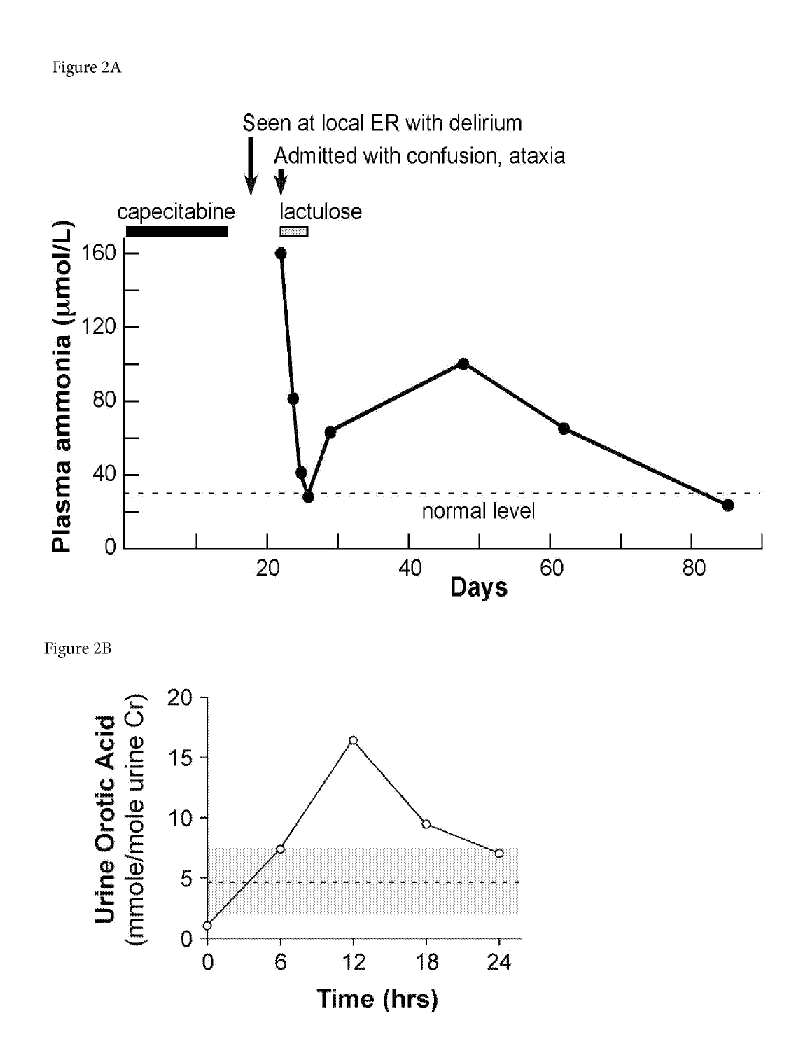

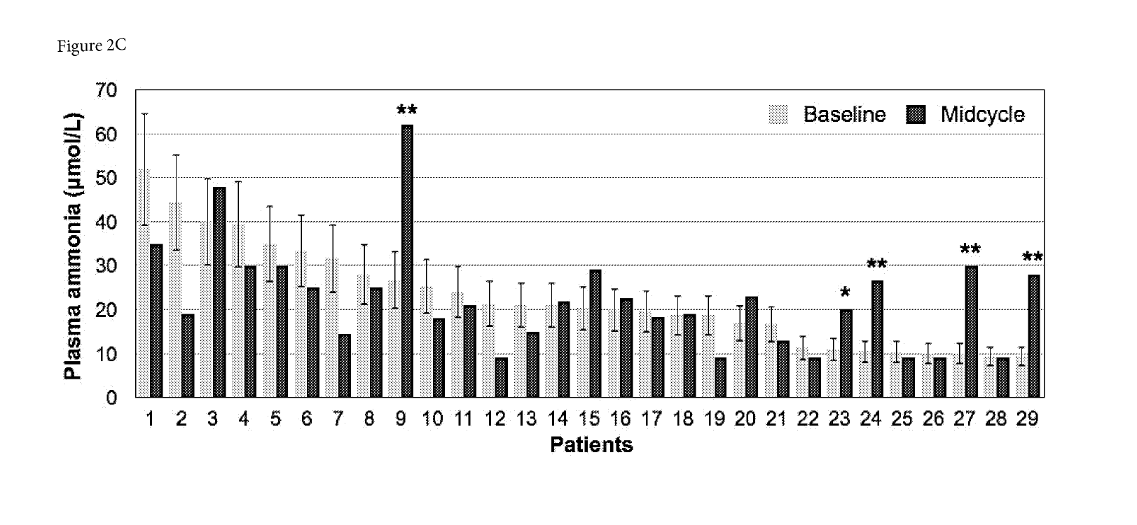

Seven days after the last capecitabine dose, she remained confused, was hospitalized, and found to have an elevated plasma ammonia level of 158 μmol/L. With lactulose treatment, plasma ammonia declined to 29 μmol/L and symptoms resolved. After discontinuation of lactulose on discharge from the hospital, plasma ammonia gradually rose and then returned to normal over two months (FIG. 2A). Four months after discharge, mild liver steatosis was noted on CT scan for the first time.

### Patient 2

A 65 y male with newly diagnosed squamous cell carcinoma of the left tonsil and base of tongue began treatment with docetaxel and cisplatin, followed by a planned 5-day infusion of 5-FU (750 mg/m²). Past medical history included manic depression treated with valproic acid.

After 1 day, the infusion 5-FU was held because of diarrhea from C. difficile, which was treated with metronidazole. Two days later, the infusion was resumed. On the third infusion day, the patient developed slurred speech and gait ataxia. On the fifth infusion day, he became delirious and then comatose. MRI and CT scan of the brain, and lumbar puncture were normal. Valproic acid trough levels (45 mcg/dL, 68 mcg/dL) were within therapeutic range, which is sufficient to inhibit N-acetylglutamate synthase (FIG. 1A) and disrupt the urea cycle (11, 12). Despite multiple episodes of diarrhea and a delay of 10 hours following discontinuation of infusion 5-FU, plasma ammonia was elevated at 37 μmol/L. The next day, the patient was alert, and plasma ammonia was 16 μmol/L. The tumor had decreased markedly in size, no longer preventing him from turning his head.

### Patient 3

A 75 y male with a well-differentiated neuroendocrine tumor of unknown primary began treatment with capecitabine (days 1-14) and temozolomide (days 10-14) after progression of massive liver metastases. Liver function tests were mildly elevated: total bilirubin 0.9 mg/dL (normal: <1.4); aspartate transaminase (AST) 80 U/L (normal: <40); alanine transaminase (ALT) 53 U/L (normal: <80); and alkaline phosphatase 1218 U/L (normal: <130).

Plasma ammonia was 59 μmol/L after 5 days of capecitabine at a dose of 500 mg twice daily, which was 50% of the intended dose. Capecitabine was doubled on day 6, because the patient had exhausted other therapeutic options for the neuroendocrine tumor. The patient was referred to us after we discovered the association of capecitabine with hyperammonemia, we instituted aggressive measures to control hyperammonemia. The lactulose dose of 15 ml twice daily was increased to three times daily, and rifaxamin 550 mg twice daily was added. On the evening of day 7, the patient became incoherent and confused. His wife considered bringing him to the emergency room, but mental status improved after a large bowel movement of soft stool. On days 8 and 12 of capecitabine, plasma ammonia was 108 μmol/L and 132 μmol/L, the patient displayed slowed speech, required assistance while ambulating, and spent most of the day in bed. Seven days after discontinuing capecitabine, plasma ammonia was 54 μmol/L, and the patient was alert, displaying normal speech, and ambulating normally.

Hypothesis for Encephalopathy after 5-FU Due to a Partially Dysfunctional Urea Cycle

The urea cycle was compromised by the urea cycle inhibitor valproic acid in Patient 2 and by massive liver metastases in Patient 3. We hypothesized that 5-FU induced hyperammonemia in Patient 1 by unmasking a partially dysfunctional urea cycle. Ammonia is eliminated by two carbamoyl phosphate synthases, CPS I, the first step in the urea cycle, and CPS II, the first step in pyrimidine biosynthesis (FIG. 1A).

CPS I localizes to mitochondria and catalyzes the reaction:

2ATP+HCO₃⁻+NH₄⁺→2ADP+carbamoyl phosphate+Pi

CPS II localizes to the cytosol and catalyzes the reaction:

Gln+CO₂+2ATP+H₂O→carbamoyl phosphate+Glu+2ADP+Pᵢ

For CPS II, ammonia is the actual substrate for the carbamoyl phosphate synthesis step, with a Kₘ for ammonia (160 μmol/L), comparable to CPS I (13). The end product of pyrimidine biosynthesis UTP inhibits CPS II (14), and the 5-FU metabolite 5-FUTP inhibits CPS II in yeast (15), and presumably in mammals. Thus, 5-FU appears to interfere with ammonia removal by inhibiting CPS II.

Evidence for More than One Defect Affecting the Urea Cycle in Patient 1

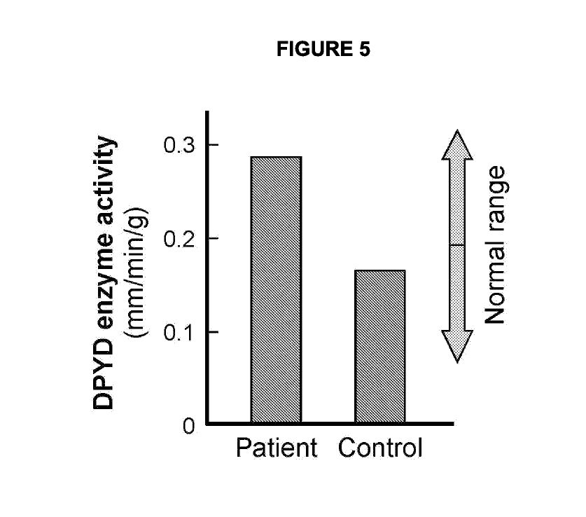

Encephalopathy (without documented hyperammonemia) has been associated with dihydropyrimidine dehydrogenase (DPYD) deficiency, which interferes with 5-FU catabolism, has been associated with 5-FU-induced encephalopathy (16, 17). In Patient 1, DPYD enzymatic activity was normal (FIG. 5), and the common mutations, DPYD*2A (IVS14+1 G>A) and DPYD*13 (1679 T>G; 1560S), were absent (Diasio Laboratory, Mayo Clinic, Rochester, Minn.).

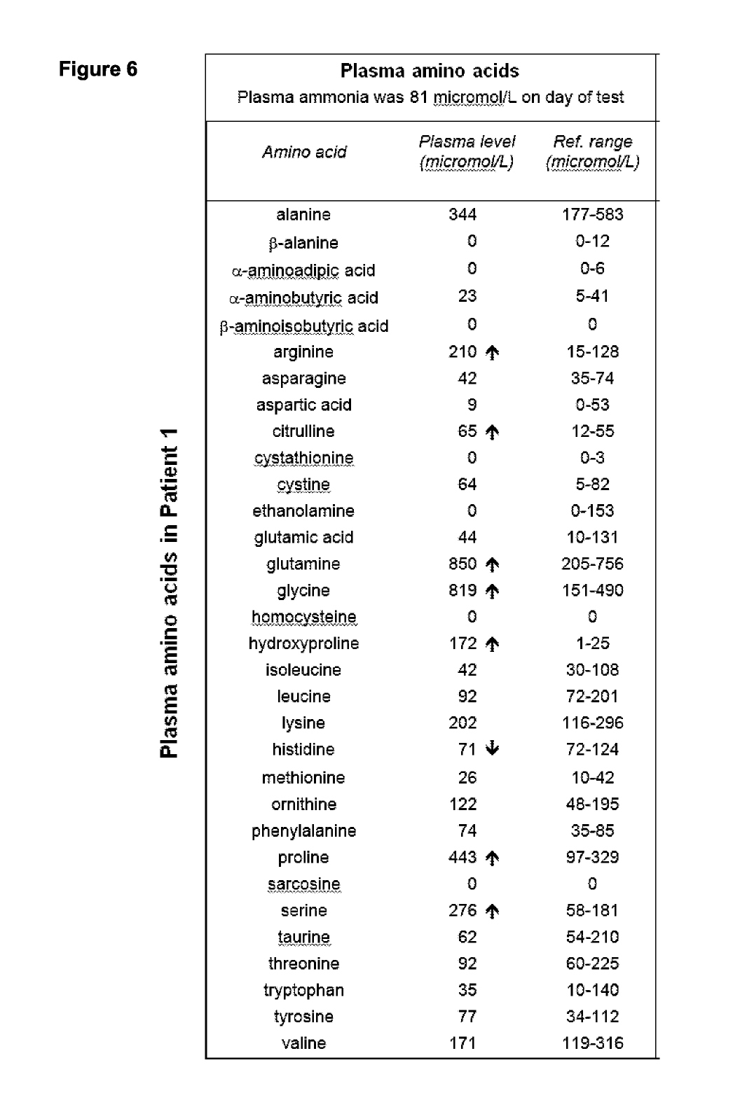

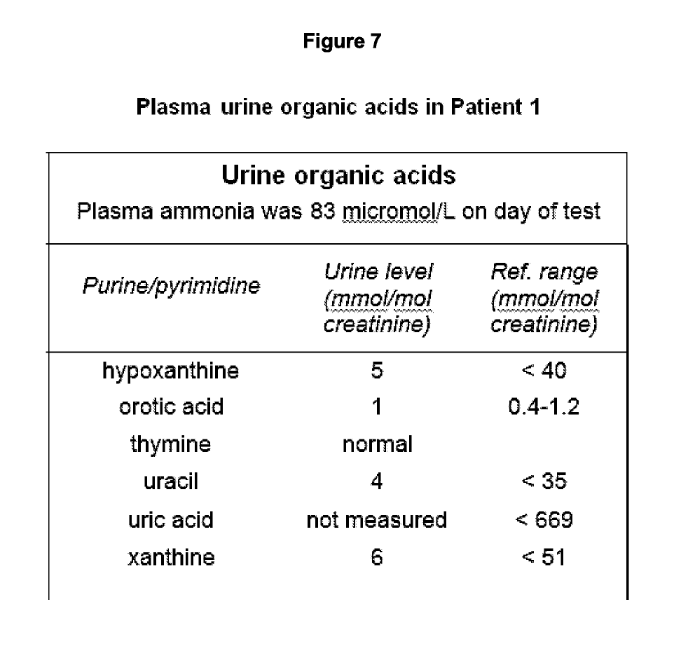

Other laboratory tests suggested that Patient 1 had more than one defect affecting the urea cycle. Plasma levels were abnormally elevated for 7 or 32 amino acids in a pattern does not correspond to a single defect in the urea cycle (FIG. 6 and FIG. 7). However, one defect involved either ornithine mitochondrial transport or ornithine transcarbamylase, since urine orotic acid was in the upper range of normal at baseline, and abnormally elevated after allopurinol challenge (16.5 nmol/mol creatinine, FIG. 2B).

### Direct and Indirect Effects of Hyperammonemia Genes on the Urea Cycle

We identified 41 genes by searching OMIM (Online Mendelian Inheritance in Man) with the keyword “hyperammonemia” and eliminating false hits. The SLC25A2 (ORNT2) and SLC25A29 (ORNT3) genes were added because they encode mitochondrial membrane transporters that act in parallel with the classical urea cycle ornithine transporter SLC25A15 (ORNT1) (18, 19). The DPYD gene was added because of its association with 5-FU-induced encephalopathy.

These 44 “hyperammonemia genes” were involved in the urea cycle, or in the apparently diverse pathways for the Krebs cycle, mitochondrial fatty acid oxidation, and several organic acidemias (Table 1). However, the non-urea cycle genes share the common feature of facilitating anaplerosis, the process that replenishes Krebs cycle intermediates. Anaplerosis appears to suppress the urea cycle by competition for glutamate and aspartate (FIG. 1). Glutamate generates either α-ketoglutarate for anaplerosis of the Krebs cycle, or N-acetylglutamate for the urea cycle. Aspartate generates either oxaloacetate for anaplerosis, or arginosuccinate for the urea cycle.

GLUD1 (glutamate dehydrogenase) deaminates glutamate to supply α-ketoglutarate to the Krebs cycle. GLUD1 is the only hyperammonemia gene with autosomal dominant inheritance. Mutations cause hyperinsulinism-hyperammonemia syndrome by generating hyperactive GLUD1, which increases ammonia production by deamination of glutamate, and decreases ammonia elimination by competing with the urea cycle for glutamate (FIG. 1A).

PC (pyruvate carboxylase) mutations disrupt conversion of pyruvate to oxaloacetate for the Krebs cycle (FIG. 1B). PC activity is also disrupted by PD (pyruvate dehydrogenase) mutations, which decrease levels of the PC co-factor acetyl-CoA. To replenish oxaloacetate for the Krebs cycle, AST activity increases, thus suppressing the urea cycle by competing for aspartate.

Fatty acid oxidation gene mutations cause proprionic acidemia and methylmalonic acidemias, and deplete succinyl-CoA in the Krebs cycle (FIG. 1B). To replenish succinyl-CoA, GLUD1 activity increases, leading to the decreased glutamate and glutamine levels observed in the proprionic and methylmalonic acidemias, and to the increased ammonia levels observed for GLUD1 mutations.

Propionic and methylmalonic acidemias also cause hyperammonemia by other mechanisms. Injection of rats with propionic or methylmalonic acid causes hyperammonemia with N-acetylglutamate depletion. Propionyl-CoA accumulates in propionic and methylmalonic acidemias and acts as a competitive inhibitor of N-acetylglutamate synthase, thus suppressing the urea cycle. Furthermore, methylmalonyl-CoA accumulates in methylmalonic acidemia and acts as a competitive inhibitor of PC, suppressing the urea cycle as described for PC mutations.

Mutations in a subset of the branched-chain amino acid degradation genes (HLCS, HMGCL, IVD, MCCC1, and MCCC2, but not the maple syrup urine disease genes, BCKDHA, BCKDHB, DBT, and DLD) cause hyperammonemia (27), probably due to accumulation of acyl-CoA intermediates of branched-chain amino acid degradation that inhibit pyruvate dehydrogenase (PD), suppressing the urea cycle as described for PD mutations.

TUFM (Tu translation elongation factor, mitochondrial) mutations cause combined oxidative phosphorylation deficiency by reduced translation of mitochondrial proteins (28). Since oxidative phosphorylation is coupled to fatty acid oxidation and the Krebs cycle, mutations suppress the urea cycle. In summary, mutations that disrupt Krebs cycle anaplerosis enzymes lead to increased activity of other anaplerosis enzymes that utilize glutamate or aspartate, thus suppressing the urea cycle.

### Deleterious Mutations in Patient 1 Causing Risk for Hyperammonemia

We analyzed the exome sequence of Patient 1 in two stages. In stage 1, we focused on the sub-exome of 44 hyperammonemia genes, and did not find overtly deleterious mutations (nonsense, invariant splice site, and insertion/deletion mutations), but did find 15 non-synonymous single nucleotide polymorphisms (SNPs) (FIG. 8). SNPs in ETFA and SLC25A2 encoded amino acid substitutions predicted to be deleterious by two methods: SIFT (Sorting Tolerant From Intolerant) based on evolutionary conservation (29); and PolyPhen-2 (Polymorphism Phenotyping version 2) based on sequence and structure-based algorithms (30).

ETFA and ETFB encode the alpha and beta subunits of ETF, an electron-transfer-flavoprotein linking acyl-CoA dehydrogenase (ACAD) to the respiratory chain in the fatty acid oxidation pathway (FIG. 1B). The SNP in ETFA encoded a T171I substitution that confers decreased thermal stability to the protein, and is over-represented in very-long-chain acyl-CoA dehydrogenase deficiency patients (31).

SLC25A2 encodes ornithine transporter ORNT2, which provides redundant function for the classical urea cycle transporter SLC25A15 (ORNT1). The SNP in SLC25A2 encoded a G159C substitution that compromises ORNT2-mediated ornithine transport when the mutant protein is expressed in tissue culture cells lacking ORNT1 (19).

Splice site SNPs did not occur in the invariant splice site positions SD1, SD2, SA-1 and SA-2, but did occur in non-invariant splice sites. Of these SNPs, the strongest candidate was a homozygous SNP in SLC7A7 in the SD-2 splice donor consensus sequence, (A/C)AG|GUPuAGU>(A/C)GG|GUPuAGU. However, the SNP occurs frequently in the general population (allele frequency 0.386), and had no effect on SLC7A7 mRNA expression (FIG. 3). Thus, the SNP in SLC7A7 was benign, and we assumed that other non-invariant splice site SNPs were also benign.

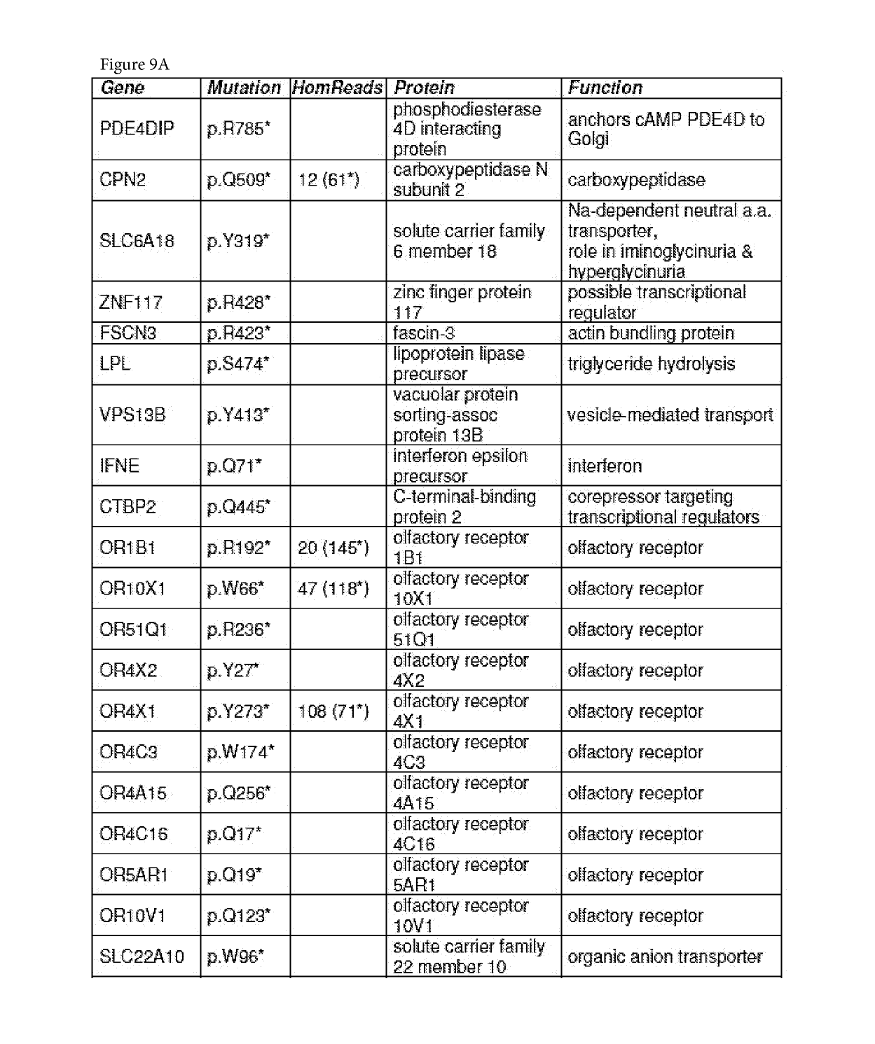

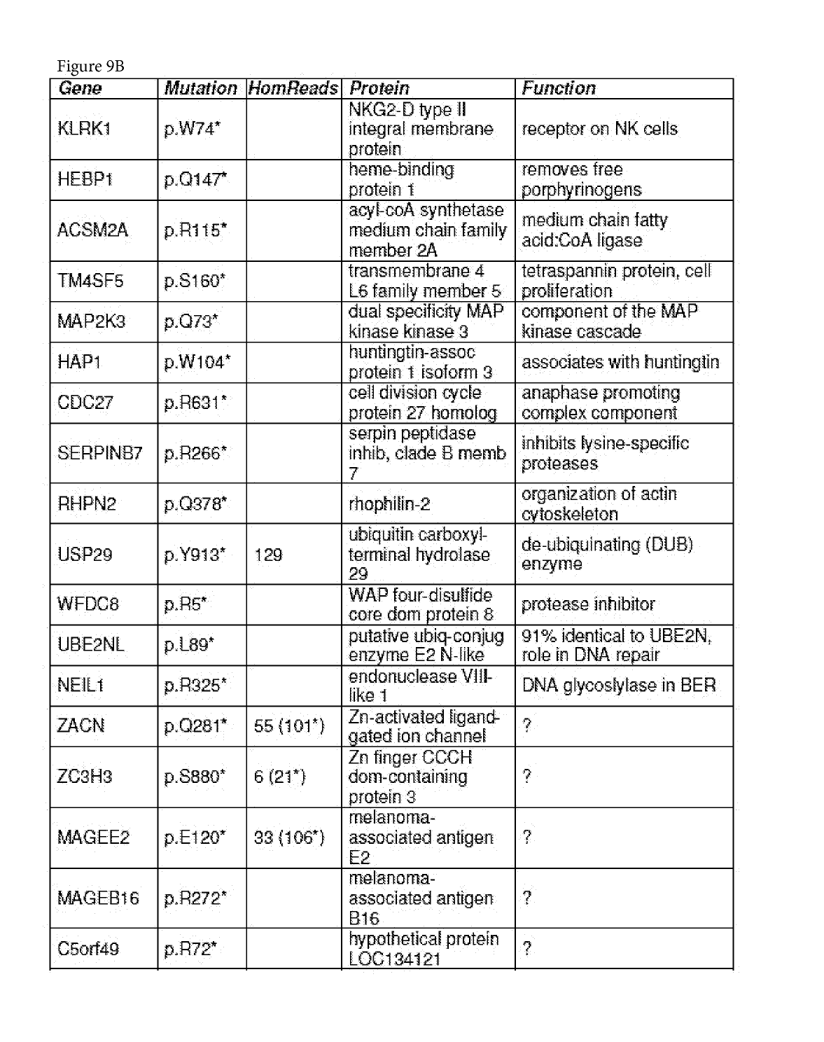

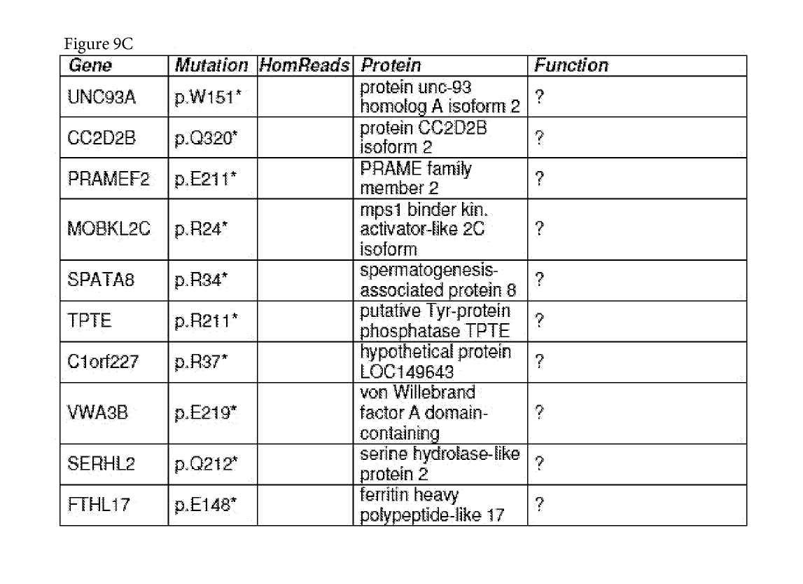

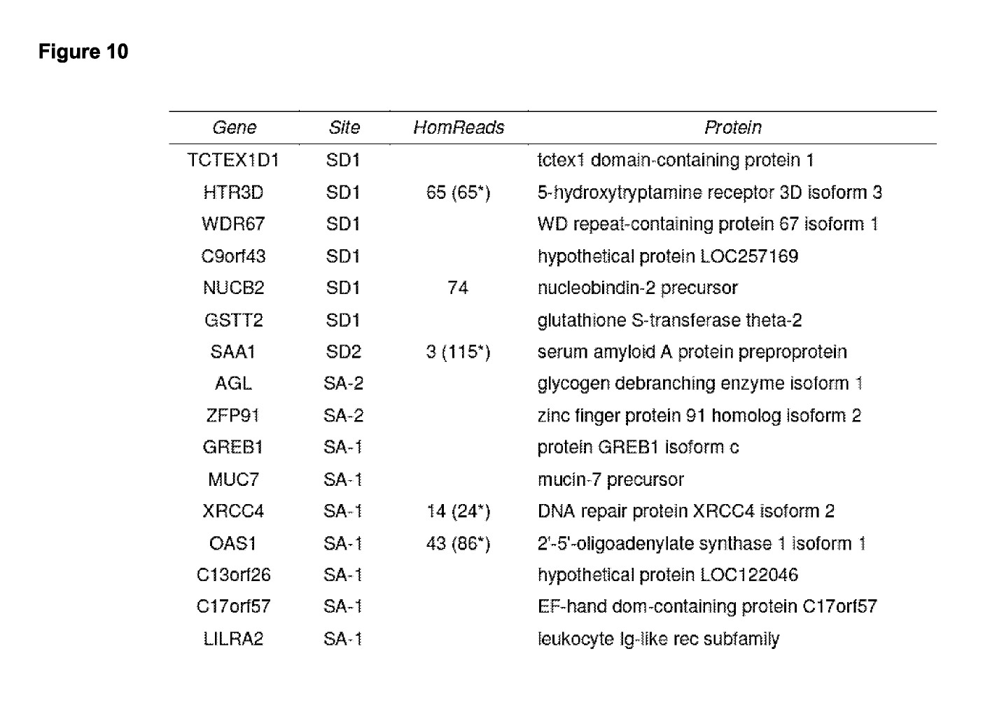

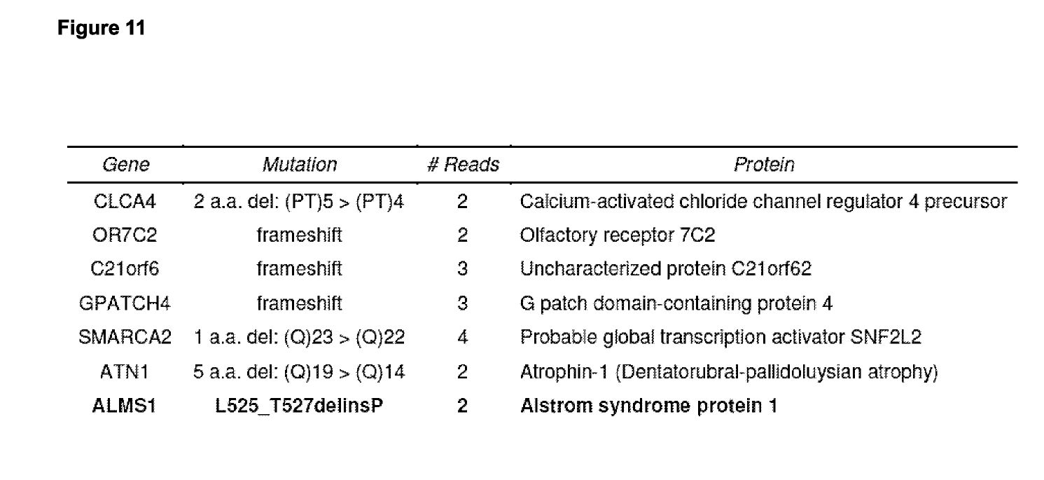

In stage 2 of the analysis, we searched the whole exome for overtly deleterious mutations in genes that were not linked to hyperammonemia in OMIM, but potentially relevant for hyperammonemia because of roles in the urea cycle or Krebs cycle anaplerosis. The whole exome contained nonsense mutations in 48 genes; invariant splice site mutations in 35 genes; and insertion/deletion mutations in 7 genes (FIG. 9, FIG. 10, FIG. 11). The ACSM2A and ALMS1 genes contained mutations relevant for hyperammonemia.

ACSM2A and its homolog ACSM2B encode acetyl-CoA synthetases, which form a thioester with CoA to activate medium chain fatty acids for beta-oxidation. Thus, ACSM2A facilitates Krebs cycle anaplerosis. In Patient 1, ACSM2A was heterozygous for nonsense mutation R115*, which generates a 462 amino acid truncation in the 577 amino acid protein.

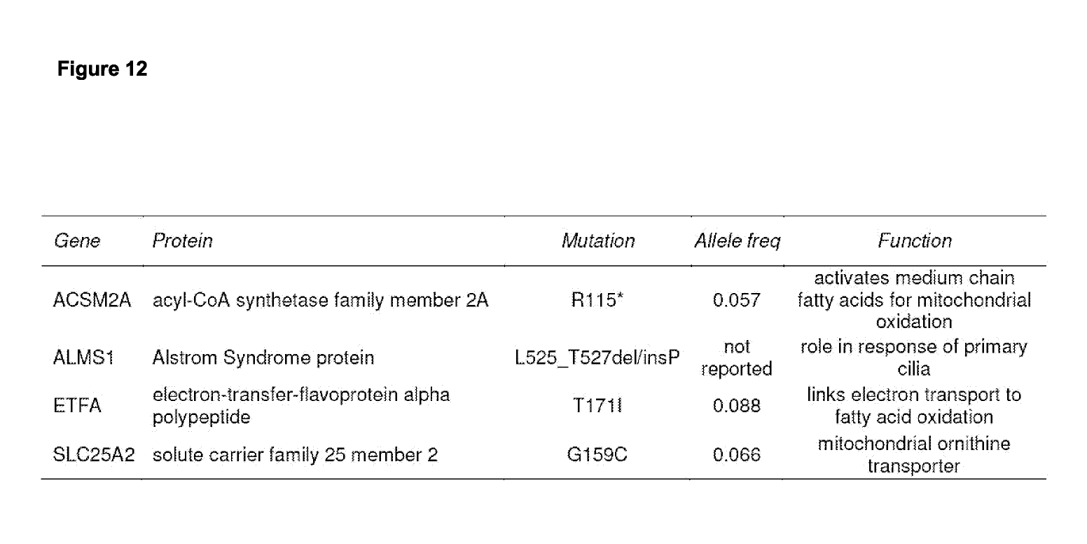

ALMS1 is mutated in autosomal recessive Alstrom Syndrome and required for the normal function of primary cilia. ALMS1 affects multiple tissues, including liver, the major site for the urea cycle. ALMS1 was heterozygous for the insertion/deletion mutation L525T527delinsP, which replaces L525, E526, and T527 with proline, for a net loss of two amino acids. This mutation was not among the 79 reported Alstrom Syndrome mutations, most of which are private mutations (35). Therefore, L525T527delinsP represents a new private mutation. Thus, Patient 1 carried one mutation disrupting ornithine transport, two mutations disrupting fatty acid oxidation (marked by stars in FIG. 1), and a fourth mutation disrupting the entire urea cycle via liver damage (FIG. 12).

### High Prevalence of Deleterious Mutations in Hyperammonemia Genes

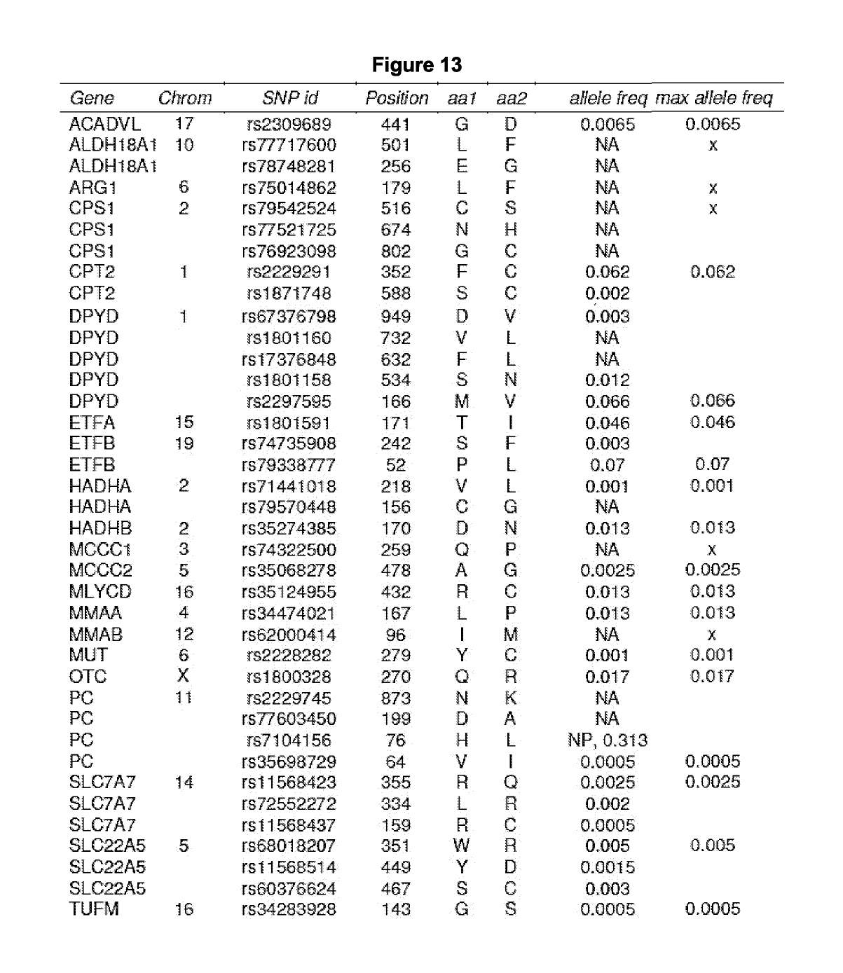

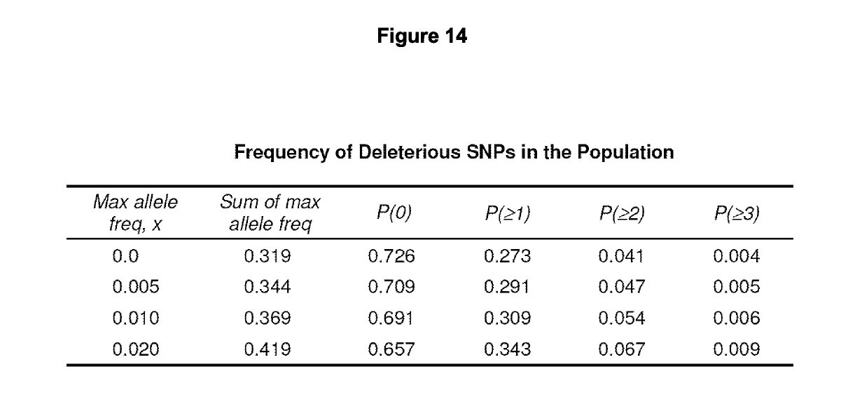

To estimate the number of deleterious mutations in the population, we screened the 44 hyperammonemia genes and found 21 genes with 39 non-synonymous SNPs predicted to be deleterious (FIG. 13). Nonsense, invariant splice site, and insertion/deletion mutations were rare. To account for linkage disequilibrium, we used the maximum allele frequency for each gene. For each of 5 genes with an unknown maximum allele frequency, x, we used the median of the 16 known maximum allele frequencies, x=0.010, corresponding to a frequency of 1%. The sum of all maximum allele frequencies, 0.369, estimated the average number of deleterious mutations in the population (FIG. 14).

Based on the Poisson probability, deleterious SNPs would occur in one or more genes in 30.9%, in two or more genes in 5.4%, and in three or more genes in 0.6% of the population. These estimates were robust, with the percentages moving up or down less than 4%, 1.5%, and 0.3%, respectively, as x varied from 0 to 0.020.

Occurrence of Hyperammonemia after Capecitabine

To prospectively measure plasma ammonia after capecitabine, we prospectively studied 29 cancer patients (Table 3). All patients had normal liver function tests, although 14 had liver metastases. Our hypothesis predicts that plasma ammonia will increase after capecitabine in some, but not all patients. To estimate baseline plasma ammonia levels, we measured 2 or more levels before the first capecitabine dose or at least 7 days after the previous capecitabine dose.

TABLE 3
Patients studied prospectively for plasma ammonia

| | | primary | | | | | |
|---|---|---|---|---|---|---|---|
| Patient | Age/ | tumor | | Other | Capecitabine | | |
| # | Sex | site | Liver | metastases | schedule | Cycle No. | Additional agents |
| 1 | 71 F | breast | Y | bone | 7/7 | 36 | bevacizumab |
| 2 | 65 M | colon | Y | lung | 14/7 | 3 | oxaliplatin, |
| | | | | | | | bevacizumab |
| 3 | 66 M | pancreas | Y | | 14/7 | 3 | temozolomide |
| | | NET | | | | | |
| 4 | 58 M | colon | Y | | 14/7 | 11 | bevacizumab |
| 5 | 69 M | pancreas | Y | | 14/7 | 3 | oxaliplatin |
| 6 | 27 F | colon | Y | | 14/7 | 7 | bevacizumab |
| 7 | 47 M | rectum | Y | | 14/7 | 1 | oxaliplatin |
| 8 | 56 M | rectum | Y | lung | 7/7 | 3 | oxaliplatin |
| 9** | 59 F | breast | Y | | 14/7 | 2 | |
| 10 | 57 M | colon | | lung | 14/7 | 3 | oxaliplatin, |
| | | | | | | | bevacizumab |
| 11 | 50 F | breast | Y | spleen | 14/7 | 4 | |
| 12 | 51 F | breast | | bone | 7/7 | 9 | |
| 13 | 81 F | stomach | | bone | 14/7 | 6 | carboplatin, |
| | | | | | | | bevacizumab |
| 14 | 51 M | pancreas | | lung | 7/7 | 5 | gemcitabine |
| 15 | 34 M | colon | | lung | 14/7 | 2 | oxaliplatin |
| 16 | 69 M | rectum | | lung | 14/7 | 12 | cetuximab |
| 17 | 56 F | colon | Y | | 14/7 | 1 | irinotecan, |
| | | | | | | | cetuximab |
| 18 | 44 F | breast | | bone | 14/7 | 3 | |
| 19 | 40 M | GE | | | 14/7 | 3 | carboplatin |
| | | junction | | | | | |
| 20 | 47 M | colon | | lung, bone | 7/7 | 14 | bevacizumab |
| 21 | 51 F | breast | | bone | 14/7 | 8 | |
| 22 | 65 M | rectum | | brain, lung | 14/7 | 5 | oxaliplatin, |
| | | | | | | | bevacizumab |
| 23* | 64 F | stomach | | | 7/7 | 5 | carboplatin |
| 24** | 57 M | unknown, | Y | | 14/14 | 3 | temozolomide |
| | | NET | | | | | |
| 25 | 74 F | breast | Y | bone, | 14/7 | 4 | |
| | | | | peritoneum | | | |
| 26 | 82 M | rectum | | lung | 7/7 | 8 | bevacizumab |
| 27** | 39 F | colon | Y | | 14/7 | 2 | oxaliplatin, |
| | | | | | | | bevacizumab |
| 28 | 70 M | unknown, | | bone, | 14/7 | 6 | |
| | | squamous | | peritoneum | | | |
| 29** | 89 M | colon | | lung | 7/7 | 1 | |

Capecitabine schedule: x/y indicates that the drug was given for x days and not given for y days.
Cycle No.: the capecitabine cycle number during which the mid-cycle plasma ammonia level was measured.
*Patients with increased plasma ammonia level in mid-cycle over baseline, p <0.01.
**Patients with increased plasma ammonia level in mid-cycle over baseline, p <0.001.
Abbreviations:
GE, gastro-esophageal;
M/F, male/female;
NET, neuroendocrine tumor;
Y, yes for liver metastases.

Several patients were not included in the study because we did not receive mid-cycle plasma ammonia levels. One such patient discontinued capecitabine during the first cycle because of severe fatigue and malaise. Because 26 of the 29 patients were enrolled after completing one or more cycles of capecitabine, enrollment was biased towards patients able to tolerate capecitabine. Thus, the study may underestimate the prevalence of severe hyperammonemia.

Mid-cycle plasma ammonia levels increased above baseline levels in 5 of the 29 patients by more than 4 standard deviations in 4 patients (corresponding to p<0.001), and more than 3 standard deviations in 1 patient (corresponding to p<0.01) (FIG. 2C). By contrast, mid-cycle plasma ammonia levels decreased below baseline levels by 2 standard deviations in 4 patients, but never by 3 standard deviations. The magnitude of baseline ammonia levels did not predict risk for increased mid-cycle levels. Two of the 5 patients, including the patient with the largest increase (Patient 5), did not receive a concurrent anticancer agent (Table 3), suggesting that the increases in plasma ammonia were attributable to capecitabine. Increased plasma ammonia can occur within the first week of treatment: Patients 23 and 29 showed increases in plasma ammonia on day 7 of capecitabine treatment; and Patient 24 showed an increase in plasma ammonia on day 4 of treatment.

We monitored changes in cognitive function between baseline and mid-cycle time points with two instruments: a telephone-administered mini-mental status examination, and a patient self-administered questionnaire. The self-administered questionnaire was adapted from a previously validated questionnaire for chronic changes in cognitive function among cancer patients undergoing chemotherapy (36).

Clinically significant symptoms occurred in 2 of the 5 patients with increased mid-cycle ammonia levels. Patient 24 attempted to work during treatment, but his office staff expressed concern about cognitive dysfunction and asked him to suspend work during the second week of each 28-day treatment cycle. Of note, he became confused, failed to complete his mid-cycle self-administered questionnaire, and forgot to donate a blood sample on day 14. On day 16, two days after his last capecitabine dose, he donated a blood sample with a plasma ammonia level of 29 μM (compared to a baseline of 11 μM), suggesting that his peak level was significantly higher. Patient 9, who experienced the largest increase in plasma ammonia, suffered from malaise, fatigue and unsteady gait, without evidence for brain metastases by MRI or leptomeningeal disease by lumbar puncture. Thus, Patients 9 and 24 suffered from symptoms consistent with capecitabine-induced hyperammonemia.

### DISCUSSION

Three index cases demonstrated that 5-FU-induced encephalopathy can occur in the setting of a dysfunctional urea cycle. Patient 1 received capecitabine and carried deleterious mutations in the ETFA, ORNT2, ACSM2A, and ALMS1 genes. ETFA and ORNT2 were among the 44 prospectively identified hyperammonemia genes. ACSM2A is involved in fatty acid oxidation, and mutations may be an unrecognized cause of hyperammonemia. The ALMS1 mutation in Patient 1 conferred a risk for liver damage. Several chemotherapy agents are known liver toxins, including 5-FU. Indeed, hepatic steatosis developed four months after the last capecitabine dose. Subsequent episodes of hyperammonemia were triggered by urinary tract infection from urea-splitting bacteria, and by enhanced gut ammonia absorption from constipation. Patient 2 received infusion 5-FU while on treatment with the urea cycle inhibitor valproic acid. Patient 3 received capecitabine while suffering from massive metastases to the liver, the primary organ for the urea cycle. Here, plasma ammonia levels increased significantly, despite aggressive pre-emptive treatment with lactulose and rifaxamin.

The ACSM2A and ETFA mutations in fatty acid oxidation explain the abnormal plasma amino acid profile in Patient 1 (FIG. 6 and FIG. 7). Defective fatty acid oxidation decreases the supply of succinyl-CoA and acetyl-CoA to the Krebs cycle (FIG. 1B). Krebs cycle anaplerosis increases GLUD1 and AST activities. GLUD1 generates ammonia, and AST depletes aspartate. Decreased aspartate blocks the conversion of citrulline to arginosuccinate in the urea cycle. Thus, anaplerosis explains the mildly elevated plasma citrulline and low normal plasma aspartate in Patient 1.

Defective fatty acid oxidation limits the availability of short chain fatty acids, suppressing glycine decarboxylation (41, 42). Since serine hydroxymethyltransferase mediates the reversible interconversion of serine and glycine, elevated plasma glycine leads to elevated plasma serine. Thus, anaplerosis explains the markedly elevated plasma glycine and serine levels in Patient 1.

Defective fatty acid oxidation also limits the availability of fatty acids that bind and activate PPAR gamma and delta, which induce arginase transcription (44), leading to elevated plasma arginine, which in turn generates elevated plasma proline and hydroxyproline. Thus, anaplerosis explains the markedly elevated plasma arginine, proline and hydroxyproline levels in Patient 1.

Patients 1, 2 and 3 suffered significant encephalopathy. In addition, 5 of 29 prospectively studied patients showed increases in plasma ammonia, with clinically recognizable symptoms occurring in 2 of the 5 patients. Thus, capecitabine/5-FU urea cycle encephalopathy (CUE) may be under-diagnosed and more common than currently appreciated. Indeed, many patients may experience a milder form of cognitive impairment that they describe as “chemobrain”.

The risk for hyperammonemia increases when the patient is heterozygous for deleterious mutations in hyperammonemia genes. As the number of mutated genes, or the severity of the mutant alleles increases, the risk for hyperammonemia increases. Deleterious mutations in multiple hyperammonemia genes are not rare, with 2 or more genes affected in 5.4% of the population, and 3 or more genes affected n 0.6% of the population. Thus, many cases of idiopathic hyperammonemia may be due to mutations in genes that affect the urea cycle. These mutations would leave healthy individuals unaffected, but cause of idiopathic hyperammonemia in cancer patients receiving chemotherapy.

Risk prediction and diagnosis of hyperammonemia are important because there are several effective treatments. Lactulose increases fecal nitrogen excretion and acidifies the stool to prevent ammonia absorption; rifaximin alters the gut flora; and sodium benzoate and sodium phenylbutyrate provide alternative pathways for urinary excretion of nitrogen. Such agents can permit continuation of chemotherapy, prevent brain damage, and improve quality of life for many patients.

### REFERENCES

1. T. A. Ahles, A. J. Saykin, Candidate mechanisms for chemotherapy-induced cognitive changes. Nat Rev Cancer 7, 192 (2007).
2. F. S. van Dam, S. B. Schagen, M. J. Muller, W. Boogerd, E. vd Wall, M. E. Droogleever Fortuyn, S. Rodenhuis, Impairment of cognitive function in women receiving adjuvant treatment for high-risk breast cancer: high-dose versus standard-dose chemotherapy. J Natl Cancer Inst 90, 210 (1998).
3. S. Deprez, F. Amant, A. Smeets, R. Peeters, A. Leemans, W. Van Hecke, J. S. Verhoeven, M. R. Christiaens, J. Vandenberghe, M. Vandenbulcke, S. Sunaert, Longitudinal assessment of chemotherapy-induced structural changes in cerebral white matter and its correlation with impaired cognitive functioning. J Clin Oncol 30, 274 (2012).
4. L. Nott, T. J. Price, K. Pittman, K. Patterson, J. Fletcher, Hyperammonemia encephalopathy: an important cause of neurological deterioration following chemotherapy. Leuk Lymphoma 48, 1702 (2007).
5. K. H. Yeh, A. L. Cheng, High-dose 5-fluorouracil infusional therapy is associated with hyperammonaemia, lactic acidosis and encephalopathy. Br J Cancer 75, 464 (1997).
6. B. Niemann, C. Rochlitz, R. Herrmann, M. Pless, Toxic encephalopathy induced by capecitabine. Oncology 66, 331 (2004).
7. A. Videnovic, I. Semenov, R. Chua-Adajar, L. Baddi, D. T. Blumenthal, A. C. Beck, T. Simuni, S. Futterer, W. Gradishar, C. Tellez, J. J. Raizer, Capecitabine-induced multifocal leukoencephalopathy: a report of five cases. Neurology 65, 1792 (2005).
8. M. Fantini, L. Gianni, D. Tassinari, S. Nicoletti, C. Possenti, F. Drudi, M. Sintini, L. Bagli, E. Tamburini, A. Ravaioli, Toxic encephalopathy in elderly patients during treatment with capecitabine: literature review and a case report. J Oncol Pharm Pract 17, 288 (2011).
9. H. Koenig, A. Patel, Biochemical basis for fluorouracil neurotoxicity. The role of Krebs cycle inhibition by fluoroacetate. Arch Neurol 23, 155 (1970).
10. A. Patel, H. Koenig, Some neurochemical aspects of fluorocitrate intoxication. J Neurochem 18, 621 (1971).
11. C. C. Aires, A. van Cruchten, L. Ijlst, I. T. de Almeida, M. Duran, R. J. Wanders, M. F. Silva, New insights on the mechanisms of valproate-induced hyperammonemia: inhibition of hepatic N-acetylglutamate synthase activity by valproyl-CoA. J Hepatol 55, 426 (2011).
12. F. X. Coude, G. Grimber, P. Parvy, D. Rabier, F. Petit, Inhibition of ureagenesis by valproate in rat hepatocytes. Role of N-acetylglutamate and acetyl-CoA. Biochem J 216, 233 (1983).
13. S. M. Shaw, E. A. Carrey, Regulation of the mammalian carbamoyl-phosphate synthetase II by effectors and phosphorylation. Altered affinity for ATP and magnesium ions measured using the ammonia-dependent part reaction. Eur J Biochem 207, 957 (1992).
14. D. R. Evans, H. I. Guy, Mammalian pyrimidine biosynthesis: fresh insights into an ancient pathway. J Biol Chem 279, 33035 (2004).
15. F. Lacroute, Regulation of pyrimidine biosynthesis in Saccharomyces cerevisiae. J Bacteriol 95, 824 (1968).
16. R. B. Diasio, T. L. Beavers, J. T. Carpenter, Familial deficiency of dihydropyrimidine dehydrogenase. Biochemical basis for familial pyrimidinemia and severe 5-fluorouracil-induced toxicity. J Clin Invest 81, 47 (1988).
17. G. Milano, M. C. Etienne, V. Pierrefite, M. Barberi-Heyob, R. Deporte-Fety, N. Renee, Dihydropyrimidine dehydrogenase deficiency and fluorouracil-related toxicity. Br J Cancer 79, 627 (1999).
18. J. A. Camacho, N. Rioseco-Camacho, The human and mouse SLC25A29 mitochondrial transporters rescue the deficient ornithine metabolism in fibroblasts of patients with the hyperornithinemia-hyperammonemia-homocitrullinuria (HHH) syndrome. Pediatr Res 66, 35 (2009).
19. J. A. Camacho, N. Rioseco-Camacho, D. Andrade, J. Porter, J. Kong, Cloning and characterization of human ORNT2: a second mitochondrial ornithine transporter that can rescue a defective ORNT1 in patients with the hyperornithinemia-hyperammonemia-homocitrullinuria syndrome, a urea cycle disorder. Mol Genet Metab 79, 257 (2003).
20. O. E. Owen, S. C. Kalhan, R. W. Hanson, The key role of anaplerosis and cataplerosis for citric acid cycle function. J Biol Chem 277, 30409 (2002).
21. C. A. Stanley, Regulation of glutamate metabolism and insulin secretion by glutamate dehydrogenase in hypoglycemic children. Am J Clin Nutr 90, 862S (2009).

22. I. Nissim, O. Horyn, B. Luhovyy, A. Lazarow, Y. Daikhin, M. Yudkoff, Role of the glutamate dehydrogenase reaction in furnishing aspartate nitrogen for urea synthesis: studies in perfused rat liver with 15N-15. Biochem J 376, 179 (2003).
23. H. R. Filipowicz, S. L. Ernst, C. L. Ashurst, M. Pasquali, N. Longo, Metabolic changes associated with hyperammonemia in patients with propionic acidemia. Mol Genet Metab 88, 123 (2006).
24. P. M. Stewart, M. Walser, Failure of the normal ureagenic response to amino acids in organic acid-loaded rats. Proposed mechanism for the hyperammonemia of propionic and methylmalonic acidemia. J Clin Invest 66, 484 (1980).
25. F. X. Coude, L. Sweetman, W. L. Nyhan, Inhibition by propionyl-coenzyme A of N-acetylglutamate synthetase in rat liver mitochondria. A possible explanation for hyperammonemia in propionic and methylmalonic acidemia. J Clin Invest 64, 1544 (1979).
26. M. C. Scrutton, M. F. Utter, Pyruvate carboxylase. IX. Some properties of the activation by certain acyl derivatives of coenzyme A. J Biol Chem 242, 1723 (1967).
27. K. A. Strauss, E. G. Puffenberger, D. H. Morton, Maple Syrup Urine Disease. (1993).
28. P. Smits, J. Smeitink, L. van den Heuvel, Mitochondrial translation and beyond: processes implicated in combined oxidative phosphorylation deficiencies. J Biomed Biotechnol 2010, 737385 (2010).
29. P. Kumar, S. Henikoff, P. C. Ng, Predicting the effects of coding non-synonymous variants on protein function using the SIFT algorithm. Nat Protoc 4, 1073 (2009).
30. I. A. Adzhubei, S. Schmidt, L. Peshkin, V. E. Ramensky, A. Gerasimova, P. Bork, A. S. Kondrashov, S. R. Sunyaev, A method and server for predicting damaging missense mutations. Nat Methods 7, 248 (2010).
31. P. Bross, P. Pedersen, V. Winter, M. Nyholm, B. N. Johansen, R. K. Olsen, M. J. Corydon, B. S. Andresen, H. Eiberg, S. Kolvraa, N. Gregersen, A polymorphic variant in the human electron transfer flavoprotein alpha-chain (alpha-T171) displays decreased thermal stability and is overrepresented in very-long-chain acyl-CoA dehydrogenase-deficient patients with mild childhood presentation. Mol Genet Metab 67, 138 (1999).
32. P. A. Watkins, D. Maiguel, Z. Jia, J. Pevsner, Evidence for 26 distinct acyl-coenzyme A synthetase genes in the human genome. J Lipid Res 48, 2736 (2007).
33. P. Rinaldo, D. Matern, M. J. Bennett, Fatty acid oxidation disorders. Annu Rev Physiol 64, 477 (2002).
34. J. D. Marshall, S. Beck, P. Maffei, J. K. Naggert, Alstrom syndrome. Eur J Hum Genet 15, 1193 (2007).
35. J. D. Marshall, E. G. Hinman, G. B. Collin, S. Beck, R. Cerqueira, P. Maffei, G. Milan, W. Zhang, D. I. Wilson, T. Hearn, P. Tavares, R. Vettor, C. Veronese, M. Martin, W. V. So, P. M. Nishina, J. K. Naggert, Spectrum of ALMS1 variants and evaluation of genotype-phenotype correlations in Alstrom syndrome. Hum Mutat 28, 1114 (2007).
36. L. Wagner, J. J. Sweet, Z. Butt, J. S. Lai, D. Cella, Measuring patient self-reported cognitive function: development of the functional assessment of cancer therapy-cognitive function instrument. J Supportive Onc 7, W32 (1990).
37. P. D. Peppercorn, R. H. Reznek, P. Wilson, M. L. Slevin, R. K. Gupta, Demonstration of hepatic steatosis by computerized tomography in patients receiving 5-fluorouracil-based therapy for advanced colorectal cancer. Br J Cancer 77, 2008 (1998).
38. P. D. King, M. C. Perry, Hepatotoxicity of chemotherapy. Oncologist 6, 162 (2001).
39. D. Zorzi, A. Laurent, T. M. Pawlik, G. Y. Lauwers, J. N. Vauthey, E. K. Abdalla, Chemotherapy-associated hepatotoxicity and surgery for colorectal liver metastases. Br J Surg 94, 274 (2007).
40. S. N. Chin, T. K. Kim, L. L. Siu, Hepatic steatosis secondary to capecitabine: a case report. J Med Case Reports 4, 227 (2010).
41. R. K. Hampson, L. L. Barron, M. S. Olson, Stimulation of the glycine cleavage system by short-chain fatty acids in isolated rat liver mitochondria. Biochemistry 23, 4604 (1984).
42. R. K. Hampson, M. K. Taylor, M. S. Olson, Regulation of the glycine cleavage system in the isolated perfused rat liver. J Biol Chem 259, 1180 (1984).
43. H. E. Xu, M. H. Lambert, V. G. Montana, D. J. Parks, S. G. Blanchard, P. J. Brown, D. D. Sternbach, J. M. Lehmann, G. B. Wisely, T. M. Willson, S. A. Kliewer, M. V. Milburn, Molecular recognition of fatty acids by peroxisome proliferator-activated receptors. Mol Cell 3, 397 (1999).
44. A. Gallardo-Soler, C. Gomez-Nieto, M. L. Campo, C. Marathe, P. Tontonoz, A. Castrillo, I. Corraliza, Arginase I induction by modified lipoproteins in macrophages: a peroxisome proliferator-activated receptor-gamma/delta-mediated effect that links lipid metabolism and immunity. Mol Endocrinol 22, 1394 (2008).
45. G. Wu, S. M. Morris, Jr., Arginine metabolism: nitric oxide and beyond. Biochem J 336 (Pt 1), 1 (1998).
46. D. V. Santi, C. S. McHenry, H. Sommer, Mechanism of interaction of thymidylate synthetase with 5-fluorodeoxyuridylate. Biochemistry 13, 471 (1974).
47. N. Segura-Bruna, A. Rodriguez-Campello, V. Puente, J. Roquer, Valproate-induced hyperammonemic encephalopathy. Acta Neurol Scand 114, 1 (2006).
48. B. C. Sharma, P. Sharma, A. Agrawal, S. K. Sarin, Secondary prophylaxis of hepatic encephalopathy: an open-label randomized controlled trial of lactulose versus placebo. Gastroenterology 137, 885 (2009).
49. N. M. Bass, K. D. Mullen, A. Sanyal, F. Poordad, G. Neff, C. B. Leevy, S. Sigal, M. Y. Sheikh, K. Beavers, T. Frederick, L. Teperman, D. Hillebrand, S. Huang, K. Merchant, A. Shaw, E. Bortey, W. P. Forbes, Rifaximin treatment in hepatic encephalopathy. N Engl J Med 362, 1071 (2010).
50. B. M. McGuire, I. A. Zupanets, M. E. Lowe, X. Xiao, V. A. Syplyviy, J. Monteleone, S. Gargosky, K. Dickinson, A. Martinez, M. Mokhtarani, B. F. Scharschmidt, Pharmacology and safety of glycerol phenylbutyrate in healthy adults and adults with cirrhosis. Hepatology 51, 2077 (2010).
51. G. M. Enns, S. A. Berry, G. T. Berry, W. J. Rhead, S. W. Brusilow, A. Hamosh, Survival after treatment with phenylacetate and benzoate for urea-cycle disorders. N Engl J Med 356, 2282 (2007).
52. J. H. Howanitz, P. J. Howanitz, C. A. Skrodzki, J. A. lwanski, Influences of specimen processing and storage conditions on results for plasma ammonia. Clin Chem 30, 906 (1984).
53. B. Maranda, J. Cousineau, P. Allard, M. Lambert, False positives in plasma ammonia measurement and their clinical impact in a pediatric population. Clin Biochem 40, 531 (2007).
54. E. Riudor, J. A. Arranz, M. Rodes, V. Rubio, M. Sentis, A. B. Burlina, Influence of dose and age on the response of the allopurinol test for ornithine carbamoyltransferase deficiency in control infants. J Inherit Metab Dis 23, 662 (2000).
55. D. L. Pearson, S. Dawling, W. F. Walsh, J. L. Haines, B. W. Christman, A. Bazyk, N. Scott, M. L. Summar, Neonatal pulmonary hypertension-urea-cycle intermediates, nitric oxide production, and carbamoyl-phosphate synthetase function. N Engl J Med 344, 1832 (2001).
56. R. M. Moonen, I. Reyes, G. Cavallaro, G. Gonzalez-Luis, J. A. Bakker, E. Villamor, The T1405N carbamoyl phosphate synthetase polymorphism does not affect plasma arginine concentrations in preterm infants. PLoS One 5, e10792 (2010).
57. P. J. Isackson, M. J. Bennett, U. Lichter-Konecki, M. Willis, W. L. Nyhan, V. R. Sutton, I. Tein, G. D. Vladutiu, CPT2 gene mutations resulting in lethal neonatal or severe infantile carnitine palmitoyltransferase II deficiency. Mol Genet Metab 94, 422 (2008).
58. V. Champattanachai, J. R. Ketudat Cairns, V. Shotelersuk, S. Keeratichamroen, P. Sawangareetrakul, C. Srisomsap, V. Kaewpaluek, J. Svasti, Novel mutations in a Thai patient with methylmalonic acidemia. Mol Genet Metab 79, 300 (2003).

What is claimed is:

1. A method of determining a susceptibility to 5-fluorouracil (5-FU) or capecitabine toxicity in a human subject, comprising:
assaying a biological sample from a human subject who has been diagnosed with cancer for the presence or absence of a deleterious polymorphism or mutation in one or more of the genes listed in Tables 1 and 2;
determining that the human subject has an increased susceptibility to 5-fluorouracil (5-FU) or capecitabine toxicity when a deleterious polymorphism or mutation in one or more of the genes listed in Tables 1 and 2 is present; and
providing an analysis indicating whether an increased susceptibility was determined.

2. The method of claim 1, further comprising extracting or isolating the biological sample from the subject prior to the step of analyzing.

3. The method of claim 1 or 2, wherein the step of assaying comprises sequencing a nucleic acid from the biological sample or sequencing a nucleic acid that has been amplified from the biological sample.

4. The method according to any of claims 1-3, wherein the step of assaying further comprises, prior to sequencing, amplification via polymerase chain reaction (PCR) of either genomic DNA or cDNA.

5. The method according to any of claims 1-4, wherein the analysis is a printed or electronic document.

6. The method according to any of claims 1-5, further comprising, after the step of determining, when an increased susceptibility to 5-fluorouracil (5-FU) or capecitabine toxicity is determined:
directing a therapeutic intervention that either:
(i) comprises administration of a reduced dose of 5-FU or capecitabine relative to an otherwise conventional dose; or
(ii) does not comprise administration of 5-FU or capecitabine.

7. The method according to any of claims 1-5, further comprising, when an increased susceptibility to 5-fluorouracil (5-FU) or capecitabine toxicity is determined:
directing a therapeutic intervention comprising:
administering 5-FU or capecitabine to the subject;
measuring the level of ammonia in the blood of the subject; and
monitoring the subject for clinical signs of 5-FU or capecitabine toxicity.

8. The method according to any of claims 1-7, wherein the biological sample is a blood sample.

9. The method according to any of claims 1-8, wherein the biological sample is assayed for the presence of a deleterious polymorphism or mutation in two or more of the genes listed in Tables 1 and 2.

10. The method according to claim 9, wherein two of the two or more genes are ETFA and SLC25A2.

11. The method according to any of claims 1-10, wherein the biological sample is assayed for the presence of a deleterious polymorphism or mutation in all of the genes listed in Table 1.

12. The method according to any of claims 1-10, wherein the biological sample is assayed for the presence of a deleterious polymorphism or mutation in at least one gene involved in Krebs cycle anaplerosis.

13. The method according to any of claims 1-12, wherein the biological sample is assayed for the presence of a deleterious polymorphism or mutation in at least one gene involved in fatty acid oxidation.

14. The method according to any of claims 1-13, wherein comprising, prior to the step of determining, at least one of:
(a) assaying the biological sample for dihydropyrimidine dehydrogenase (DPYD) enzymatic activity; and
(b) assaying the biological sample for the presence of a deleterious polymorphism or mutation in DPYD.

15. A system for determining a susceptibility to 5-fluorouracil (5-FU) or capecitabine toxicity in a human subject, the system comprising:
(i) a genotype determination element for determining the presence or absence in a biological sample of a deleterious polymorphism or mutation in one or more of the genes listed in Tables 1 and 2; and
(ii) a prognosis analysis element for guiding a course of treatment based on the determined presence or absence of a deleterious polymorphism or mutation.

16. A method of treating a human subject based on a determined susceptibility to 5-fluorouracil (5-FU) or capecitabine toxicity, the method comprising:
(a) assaying a biological sample from a human subject who has been diagnosed with cancer for the presence of a deleterious polymorphism or mutation in one or more of the genes listed in Tables 1 and 2;
(b) determining an increased susceptibility to 5-fluorouracil (5-FU) or capecitabine toxicity for the subject when a deleterious polymorphism or mutation is present in the biological sample; and
(c) directing a therapeutic intervention other than administration of 5-FU or capecitabine when an increased susceptibility to 5-fluorouracil (5-FU) or capecitabine toxicity is determined.
